SEQUENCE LISTING (10 sequences)

SEQ ID NO: 1 - protein, 162 aa, Artificial Sequence
  Amino acid sequence identified using molecular biology techniques.
GEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 2 - DNA, 489 bp, Artificial Sequence
  Nucleic acid sequence identified using molecular biology techniques.
cacggtgaaggtacgttcacctctgacctgagcaaacagatggaggaagaagcggttcgtctgttcatcgagtggctgaaaaacggtggtccgtcttctggtgctccgccgccgtctggtggtggtggtggttctggtggtggtggttctggtggtggtggcagcgatatccagatgactcagtccccgtcttctctctccgcctctgttggcgaccgtgttaccatcacttgtcgtgcgagccagtggatcggttcccagctgagctggtatcagcagaaaccgggcaaagcgccgaaactgctgatcatgtggcgctctagcctgcagtctggtgtaccgtctcgtttctccggctctggttctggtacggacttcaccctcacgatctcttccctgcagccggaagactttgccacctactactgcgcacagggtctgcgtcacccgaaaaccttcggtcagggtaccaaagtcgagatcaaacgt

SEQ ID NO: 3 - protein, 108 aa, Artificial Sequence
  Amino acid sequence identified using molecular biology techniques.
DIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKC

SEQ ID NO: 4 - DNA, 324 bp, Artificial Sequence
  Nucleic acid sequence identified using molecular biology techniques.
gatatccagatgacccagtctccgtcttccctgtctgcgtctgttggtgatcgcgttaccatcacttgccgtgcaagccgtccgatcggtactatgctgagctggtaccagcagaaaccgggtaaagcgccgaaactgctgattctggctttctctcgcctgcagtctggtgttccgtctcgtttcagcggtagcggttctggtaccgacttcaccctgaccatttcctctctgcagccggaagacttcgctacctactattgtgcgcaggcaggtactcacccgactaccttcggtcagggcaccaaagttgaaatcaaatgc

SEQ ID NO: 5 - protein, 230 aa, Artificial Sequence
  Amino acid sequence identified using molecular biology techniques.
EVQLLESGGGLVQPGGSLRLSCAASGFTFDKYSMGWVRQAPGKGLEWVSQISDTADRTYYAHAVKGRFTISRDNSKNTLYLQMNSLRAEDTAVYYCAIYTGRWVPFEYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 6 - DNA, 690 bp, Artificial Sequence
  Nucleic acid sequence identified using molecular biology techniques.
gaggtacagctgctggaatctggtggtggtctggttcagccgggtggctctctgcgtctgtcttgtgcagcgtctggtttcaccttcgacaaatactctatgggctgggttcgtcaggcgccgggtaaaggtctggaatgggtgtctcagatctctgacaccgcagatcgtacctactacgcacacgctgtgaaaggtcgcttcaccatctctcgcgacaactccaaaaacaccctgtacctgcagatgaactccctgcgtgctgaagacaccgcggtatactattgcgcgatctacaccggtcgttgggttccgttcgaatactggggtcagggtaccctggttactgtgagctctgcgtctaccgacatccagatgacccagtctccgtcttctctgtctgcgagcgttggtgaccgtgttaccatcacttgccgtgcttctcgtccgatcggtaccactctgagctggtatcagcagaaaccgggcaaagcgccgaaactgctgatcctgtggaactctcgtctgcagtccggtgttccgtctcgtttctctggcagcggttctggtaccgacttcaccctgactatctctagcctgcagccggaagacttcgcaacctactattgcgcacaggctggtactcacccgaccactttcggtcagggtaccaaagtagaaatcaaacgt

SEQ ID NO: 7 - protein, 119 aa, Artificial Sequence
  Amino acid sequence identified using molecular biology techniques.
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 8 - DNA, 357 bp, Artificial Sequence
  Nucleic acid sequence identified using molecular biology techniques.
gaagtacaactgctggagagcggtggcggcctggttcaaccgggtggttccctgcgcctgtcctgtgcggcatctggtttcaccttcgcacacgaaaccatggtgtgggttcgccaagctccgggcaaaggcctggaatgggtaagccacattcctccagatggccaggacccattctatgcggattccgttaagggtcgctttaccatttctcgtgataactccaaaaacaccctgtacctgcagatgaactccctgcgcgccgaggatactgcggtgtaccattgtgcgctgctgcctaaacgtggcccgtggttcgattactggggtcagggtactctggtcaccgtaagcagc

SEQ ID NO: 9 - protein, 120 aa, Artificial Sequence
  Amino acid sequence identified using molecular biology techniques.
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSSA

SEQ ID NO: 10 - DNA, 360 bp, Artificial Sequence
  Nucleic acid sequence identified using molecular biology techniques.
gaagtacaactgctggagagcggtggcggcctggttcaaccgggtggttccctgcgcctgtcctgtgcggcatctggtttcaccttcgcacacgaaaccatggtgtgggttcgccaagctccgggcaaaggcctggaatgggtaagccacattcctccagatggccaggacccattctatgcggattccgttaagggtcgctttaccatttctcgtgataactccaaaaacaccctgtacctgcagatgaactccctgcgcgccgaggatactgcggtgtaccattgtgcgctgctgcctaaacgtggcccgtggttcgattactggggtcagggtactctggtcaccgtaagcagcgcg

(19) United States
(12) Patent Application Publication
(10) Pub. No.: US 2015/0368292 A1
(43) Pub. Date: Dec. 24, 2015

(54) METHOD OF PRODUCING A PROTEIN

(71) Applicant: GLAXO GROUP LIMITED, Middlesex (GB)

(72) Inventors: Alex CHATEL, London (GB); Michael HOARE, London (GB); Peter KUMPALUME, Stevenage (GB); Jessica Rachel MOLEK, King of Prussia, PA (US); Jason Michael RECK, King of Prussia, PA (US); Andrew David WEBER, King of Prussia, PA (US)

(73) Assignee: GLAXO GROUP LIMITED, Middlesex (GB)

(21) Appl. No.: 14/764,593

(22) PCT Filed: Jan. 29, 2014
(86) PCT No.: PCT/EP2014/051705

§ 371 Date: Jul. 30, 2015

Related U.S. Application Data
(60) Provisional application No. 61/758,870, filed on Jan. 31, 2013.
(60) Provisional application No. 61/899,437, filed on Nov. 4, 2013.

(51) Int. Cl. C07K 1/30; C07K 16/28; C07K 16/18; C07K 1/36

(52) U.S. Cl. CPC C07K 1/303; C07K 1/36; C07K 16/2878; C07K 16/18; C07K 2319/02; C07K 2317/569

(57) ABSTRACT

The present invention relates to a method of producing a recombinant protein by harvesting a microbial cell broth and adding an amount of a flocculant to achieve an effective particle size distribution. The present invention also relates to a method of clarifying a microbial harvest by adding an amount of a flocculant to achieve an effective particle size distribution.

The present invention relates to a method of producing a recombinant protein by harvesting a microbial cell broth and adding an amount of a flocculant to achieve an effective particle size distribution. The present invention also relates to a method of clarifying a microbial harvest by adding an amount of a flocculant to achieve an effective particle size distribution.

### BACKGROUND OF THE INVENTION

Large-scale manufacture of recombinant proteins is an important challenge for the biotechnology industry. Recombinant proteins are usually produced by host cell culture or via cell free systems. In each case, the protein is purified from a sample comprising impurities to a purity sufficient for use as a human therapeutic product. Typical processes involve initial clarification to remove solid particulates, followed by purification to ensure adequate purity. Clarification can lower the burden on subsequent chromatographic steps during purification.

Typical clarification steps comprise a centrifugation step, or a filtration step, or both. Prior to clarification, a pre-treatment step may be used as a method of conditioning the sample. An example of a conditioning pre-treatment step is flocculation which causes solid particulates to form larger aggregates which are then removed by clarification.

Much of the focus on the use of flocculants is to increase the particle size of the solid particulates present in the sample to improve the efficiency of clarification. This is because larger aggregates are easier to remove by centrifugation.

The development of a clarification method typically involves choosing an effective amount of flocculant to (i) maximise solid particulate removal, (ii) preserve product quality and product recovery, (iii) minimise the amount of flocculant used (too much causes turbidity), (iv) minimise impact of flocculant on subsequent purification steps (eg chromatographic steps), and (v) ensuring removal of flocculant to acceptable levels in the therapeutic product.

Thus, a careful balance must be struck when choosing an effective amount of flocculant to achieve the desired effects whilst minimising the undesired effects.

Empirical testing to determine an effective amount of flocculant is usually carried out at various stages of the clarification and purification processes, including one or a combination of assessing (a) floc characteristics such as (i) formation of floc (initiation of flocculation) and breakage of floc; (ii) floc size; (iii) mechanical stability/strength of floc; (iv) surface shear resistance of floc; (b) clarification efficiency; (c) filterability; and (d) purification. Such empirical testing can be time consuming and laborious.

Thus, a need exists for a more efficient method of clarification of a microbial cell harvest producing a recombinant protein.

### SUMMARY OF THE INVENTION

The present invention provides a method of producing a recombinant protein, wherein the method comprises:
(a) harvesting a microbial cell broth that expresses the recombinant protein; and
(b) adding an amount of a flocculant to achieve a particle size distribution by volume of about 5% or less particles in the size range of 5 μm or less.

In another aspect, the present invention provides a method of producing a recombinant protein, wherein the method comprises:
(a) harvesting a microbial cell broth that expresses the recombinant protein;
(b) adding an amount of a flocculant to achieve a particle size distribution by volume of about 5% or less particles in the size range of 5 μm or less; and
(c) clarifying the flocculated harvest.

In another aspect, the present invention provides a method of producing a recombinant protein, wherein the method comprises:
(a) harvesting a microbial cell broth that expresses the recombinant protein;
(b) adding an amount of a flocculant to achieve a particle size distribution by volume of about 5% or less particles in the size range of 5 μm or less;
(c) clarifying the flocculated harvest; and
(d) purifying the recombinant protein from the clarified flocculated harvest.

In another aspect, the present invention provides a method of clarifying a microbial harvest, wherein the method comprises:
(a) harvesting a microbial cell broth;
(b) adding an amount of a flocculant to achieve a particle size distribution by volume of about 5% or less particles in the size range of 5 μm or less; and
(c) clarifying the flocculated harvest.

In a further aspect, the present invention provides a modified Escherichia coli cell harvest wherein:
(a) the cells express a periplasmic targeted recombinant protein;
(b) the harvest comprises 0.01-2% PEI; and
(c) the particle size distribution by volume of the harvest is about 5% or less particles in the size range of 5 μm or less.

### BRIEF DESCRIPTION OF THE FIGURES

FIG. 1: Particle size distribution is shown for DOM100 harvest, and with the addition of 0.005%, 0.05%, 0.1%, 0.5% and 2% PEI.

FIG. 2: Percentage volume of particles equal to or less than 5 μm in diameter for Dat06 harvest, and with the addition of 0.03%, 0.05%, 0.1%, 0.5% and 2.0% PEI.

FIG. 3: Particle size distribution for DOM101 harvest (open circle) and harvest exposed to high shear (closed circle). The size distributions are presented as (a) total volume particle size distribution (log scale); particle size distributions emphasizing peak 1 (insert b), peaks 1 and 2 (insert c), and peak 3 (insert d).

FIG. 4: Particle size distribution for DOM101 harvest treated with 0.5% PEI (closed circle) and PEI flocculated harvest treated with low shear (cross) and high shear (open circle). The size distributions are presented as (a) total volume particle size distribution (log scale); particle size distributions emphasizing peak 1 (insert b), peak 1 (insert c), and peak 2 (insert d).

FIG. 5: Effect of PEI concentration on DOM100 microbial broth harvest turbidity (feed turbidity), and post-centrifugation turbidity (centrate turbidity).

FIG. 6: Ultra-scaled down model of % solids remaining for DOM0101 harvest (a) and DOM101 harvest in the presence of 0.5% PEI (b). Also represented is the sample subjected to no shear (closed circle), low shear (cross), and high shear (open circle).

FIG. 7: Effect of PEI concentration on primary filter capacity of DOM100 harvest centrate.

FIG. 8: Effect of three different flocculants on DNA concentrations in harvests for exemplar proteins Dat06 and DOM100.

FIG. 9: Effect of 0.5% PEI on filterability of exemplar protein DOM0101 harvest centrate.

FIG. 10: Variation of Vₘₐₓ in filterability of DOM0101 harvest centrate with and without 0.5% PEI treatment at various harvest post induction times.

FIG. 11: Particle size distribution for thawed DOM101 harvest (open circle), and thawed harvest treated with high shear (closed circle). The size distributions are presented as (a) total volume particle size distribution (log scale); particle size distributions emphasizing peak 1 (insert b), peaks 1, 2 and 3 (insert c), and peaks 3 and 4 (insert d).

FIG. 12: Particle size distribution for thawed DOM101 harvest (closed circle), and 0.5% PEI thawed harvest treated with high shear (open circle). The size distributions are presented as (a) total volume particle size distribution (log scale); particle size distributions emphasizing sub-peak (insert b), peak 1 (insert c), peaks 1 and 2 (insert d), and trail end of peak 2 (insert e).

FIG. 13: Particle size distribution for thawed DOM101 harvest treated with 0.5% PEI in the presence of no (closed circle), low (cross) and high shear (open circle). The size distributions are presented as (a) total volume particle size distribution (log scale); particle size distributions emphasizing peak 1 (insert b), peak 1 (insert c), and peak 2 (insert d).

FIG. 14: Particle size distribution for sheared thawed DOM101 harvest (closed circle) and homogenised thawed DOM101 harvest (open circle). The size distributions are presented as (a) total volume particle size distribution (log scale); particle size distributions emphasizing peak 1 (insert b), peaks 2 and 3 (insert c), and peak 3 (insert d).

FIG. 15: Microscopy images of thawed DOM101 harvest (a) with addition of PEI (b) and subsequent exposure to low (c) or high (d) shear.

FIG. 16: Ultra scale down model of % solids remaining for DOM0101 homogenised thawed harvest (a), DOM101 thawed harvest (b), and 0.5% PEI flocculated DOM101 thawed harvest (c) (legend as for FIG. 6).

FIG. 17: A DAT06 fermentation harvest with a concentration range of PEI 0 to 0.6% and a pH range of pH4-9 was assessed for (A) supernatant turbidity as measured at A600 nm wavelength to assess solution clarity (scale of 0.2-2.0); and (B) processibility as measured by direct filtration performance through a 0.2 μm filter under a centrifuge force (filtrate volume on a scale of 0-250).

FIG. 18: Dat06 harvest was treated with 0.1% PEI (low flocculant concentration) and 0.4% PEI (high flocculant concentration) and NaCl solutions of varying ionic strength (conductivity). “Low flocculant” and “high flocculant” used simply for comparative reasons. The mean particle diameter (μm) was assessed in A; and the % particles ≦5 μm by volume in B.

FIG. 19: DOM100 harvest was flocculated with 4.3% CaCl₂, 0.1% PEI and 0.2% PEI. Mean particle diameter was assessed in A, and % particles ≦5 μm by volume in B (particle size shown by open squares). Filter capacity was determined using a batch centrifuge and a tubular bowl centrifuge (continuous centrifuge).

FIG. 20: Dat06 and DOM100 harvests with 0.4% PEI addition were compared to the samples that were not treated with a flocculant. Clarification was then performed by centrifugation and HCP levels were measured using in house analytical immunoassays.

### DETAILED DESCRIPTION

The present invention involves the realisation that a more efficient method of clarification with a flocculant can be achieved by influencing the particle size distribution and the proportion of particles that are 5 μm or below. The inventors have realised that the proportion of particles that are 5 μm or below upon flocculant addition is determinative of clarification efficiency. By achieving a particle size distribution by volume of about 5% or less particles in the size range of 5 μm or less upon flocculant addition results in a more efficient clarification method.

Use of this method prevents the requirement for the laborious empirical testing to determine an effective amount of flocculant at various stages of the clarification process.

The methods described herein result in reduced solids content (increased solids removal) following centrifugation during clarification, when compared to no addition of a flocculant, or an amount of a flocculant that does not achieve a particle size distribution by volume of about 5% or less particles in the size range of 5 μm or less. Efficient removal of solids in this centrifugation step represents a significant benefit as improved performance has an amplified effect on downstream filtration and/or purification steps. This is also of use with cell cultures that are particularly viscous or of high density. This can result in an improved processing time through the centrifuge.

The methods described result in improved filterability, during clarification, when compared to no addition of a flocculant, or an amount of a flocculant that does not achieve a particle size distribution by volume of about 5% or less particles in the size range of 5 μm or less. This can result in increased flow rate through the filter. Also, the maximum filter capacity can be increased. Thus there is a decrease in total processing time. As a result of these advantages, filter costs can be reduced.

The methods described result in reduced turbidity following centrifugation during clarification, when compared to no addition of a flocculant, or an amount of a flocculant that does not achieve a particle size distribution by volume of about 5% or less particles in the size range of 5 μm or less.

The methods described result in reduced DNA concentration in the clarified flocculated harvest, when compared to no addition of a flocculant, or an amount of a flocculant that does not achieve a particle size distribution by volume of about 5% or less particles in the size range of 5 μm or less.

Other improvements include improved protection against the effects of shear during clarification, when compared to no addition of a flocculant.

The improvements described are also applicable to harvests that have been pre-treated by freeze-thaw and/or homogenisation.

The methods described result in identification of the minimal effective amount of flocculant to achieve the desired effects during clarification.

“About” as used herein when referring to a measurable value such as an amount, a temporal duration, and the like, is meant to encompass variations of ±1%, ±0.75%, ±0.5%, ±0.25%, ±0.2%, and ±0.1% from the specified value, as such variations are appropriate to perform the methods described.

### Recombinant Protein

The recombinant protein may comprise an antigen binding protein, a monoclonal antibody, an antibody fragment, or a domain antibody.

The recombinant protein may comprise a viral protein, a bacterial toxin, a bacterial toxoid, or a cancer antigen. For example, the bacterial toxoid is a diphtheria toxoid, such as CRM197; or a Streptococcus pneumoniae capsular saccharide conjugate and a protein component comprising Protein E and/or PilA from Haemophilus influenzae.

As used herein a “recombinant protein” refers to any protein and/or polypeptide that can be administered to a mammal to elicit a biological or medical response of a tissue, system, animal or human. The recombinant protein may elicit more than one biological or medical response. Furthermore, the term “therapeutically effective amount” means any amount which, as compared to a corresponding subject who has not received such amount, results in, but is not limited to, healing, prevention, or amelioration of a disease, disorder, or side effect, or a decrease in the rate of advancement of a disease or disorder. The term also includes within its scope amounts effective to enhance normal physiological function as well as amounts effective to cause a physiological function in a patient which enhances or aids in the therapeutic effect of a second pharmaceutical agent.

The term “antigen binding protein” as used herein refers to antibodies, antibody fragments and other protein constructs, such as domains, which are capable of binding to an antigen.

The term “antibody” is used herein in the broadest sense to refer to molecules with an immunoglobulin-like domain. As used herein, “immunoglobulin-like domain” refers to a family of polypeptides which retain the immunoglobulin fold characteristic of antibody molecules, which contain two β-sheets and, usually, a conserved disulphide bond. This family includes monoclonal (for example IgG, IgM, IgA, IgD or IgE), recombinant, polyclonal, chimeric, humanised, bispecific and heteroconjugate antibodies; a single variable domain, a domain antibody, antigen binding fragments, immunologically effective fragments, Fab, F(ab′)₂, Fv, disulphide linked Fv, single chain Fv, diabodies, TANDABS™, etc (for a summary of alternative “antibody” formats see Holliger and Hudson, Nature Biotechnology, 2005, Vol 23, No. 9, 1126-1136).

The phrase “single variable domain” refers to an antigen binding protein variable domain (for example, V_{H}, V_{HH}, V_{L}) that specifically binds an antigen or epitope independently of a different variable region or domain. A “domain antibody” or “dAb” may be considered the same as a “single variable domain” which is capable of binding to an antigen or epitope. The term “epitope-binding domain” refers to a domain that specifically binds an antigen or epitope independently of a different domain.

As used herein “domain” refers to a folded protein structure which retains its tertiary structure independently of the rest of the protein. Generally, domains are responsible for discrete functional properties of proteins and in many cases may be added, removed or transferred to other proteins without loss of function of the remainder of the protein and/or of the domain. By single antibody variable domain or immunoglobulin single variable domain is meant a folded polypeptide domain comprising sequences characteristic of an antibody variable domain. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

A domain antibody can be present in a format (e.g, homo- or hetero-multimer) with other variable regions or variable domains where the other regions or domains are not required for antigen binding by the single immunoglobulin variable domain (i.e., where the immunoglobulin single variable domain binds antigen independently of the additional variable domains).

The domain antibody may be a human antibody variable domain. The dAb may be of human origin. In other words, the dAb may be based on a human Ig framework sequence.

As used herein, the term “antigen binding site” refers to a site on an antigen binding protein which is capable of specifically binding to an antigen, this may be a single domain, or it may be paired VH/VL domains as can be found on a standard antibody. Single-chain Fv (ScFv) domains can also provide antigen-binding sites.

The antigen binding protein may comprise additional antigen binding sites for different antigens, such as additional epitope binding domains. For example, the antigen binding protein may have specificity for more than one antigen, for example two antigens, or for three antigens, or for four antigens.

The antigen binding protein may consist of, or consist essentially of, an Fc region of an antibody, or a part thereof, linked at each end, directly or indirectly (for example, via a linker sequence) to a binding domain. Such an antigen binding protein may comprise two binding domains separated by an Fc region, or part thereof. By separated is meant that the binding domains are not directly linked to one another, and may be located at opposite ends (C and N terminus) of an Fc region, or any other scaffold region.

The antigen binding protein may comprise two scaffold regions each bound to two binding domains, for example at the N and C termini of each scaffold region, either directly or indirectly via a linker. Each binding domain may bind to a different antigen.

The antigen binding protein may take the protein scaffold format of a mAbdAb. “mAbdAb” and “dAbmAb” are used interchangeably, and are intended to have the same meaning as used herein. Such antigen-binding proteins comprise a protein scaffold, for example an Ig scaffold such as IgG, for example a monoclonal antibody, which is linked to a further binding domain, for example a domain antibody. A mAbdAb has at least two antigen binding sites, at least one of which is from a domain antibody, and at least one is from a paired VH/VL domain.

Domain antibodies can exist and bind to target in monomeric or multimeric (eg dimeric) forms, and can be used in combination with other molecules for formatting and targeting approaches. For example, an antigen-binding protein having multiple domains can be made in which one of the domains binds to serum proteins such as albumin. Domain antibodies that bind serum albumin (AlbudAbs™) are described, for example, in WO05/118642 and can provide the domain fusion partner an extended serum half-life in its own right.

dAbs may also be conjugated to other molecules, for instance in the form of a dAb-conjugate or a dAb-fusion with other molecules e.g. a drug, another protein, an antibody molecule or an antibody fragment. For example a dAb can be present as a formatted dAb, e.g. the dAb can be present as a dAb-Fc fusion or conjugate as described in for example WO 2008/149148. Alternatively, the formatted dAb can be present as a mAbdAb, as described in WO 2009/068649. The dAb may be present as a fusion or conjugate with half life extending proteins or polypeptides, for example, a further dAb which binds to serum albumin (AlbudAb™), or to a half life extending chemical moiety such as polyethyleneglycol (PEG). The dAb may be present as a fusion or conjugate with further therapeutic or active molecules.

As used herein, “drug” refers to any compound (for example, a small organic molecule, a nucleic acid, a polypeptide) that can be administered to an individual to produce a beneficial therapeutic or diagnostic effect through binding to and/or altering the function of a biological target molecule in the individual. The target molecule can be an endogenous target molecule encoded by the individual's genome (eg, an enzyme, receptor, growth factor, cytokine encoded by the individual's genome) or an exogenous target molecule encoded by the genome of a pathogen. The drug may be a dAb or mAb.

A “dAb conjugate” refers to a composition comprising a dAb to which a drug is chemically conjugated by means of a covalent or noncovalent linkage. Preferably, the dAb and the drug are covalently bonded. Such covalent linkage could be through a peptide bond or other means such as via a modified side chain. The noncovalent bonding may be direct (e.g., electrostatic interaction, hydrophobic interaction) or indirect (e.g., through noncovalent binding of complementary binding partners (e.g., biotin and avidin), wherein one partner is covalently bonded to drug and the complementary binding partner is covalently bonded to the dAb). When complementary binding partners are employed, one of the binding partners can be covalently bonded to the drug directly or through a suitable linker moiety, and the complementary binding partner can be covalently bonded to the dAb directly or through a suitable linker moiety.

As used herein, “dAb fusion” refers to a fusion protein that comprises a dAb and a polypeptide drug (which could be a polypeptide, a dAb or a mAb). The dAb and the polypeptide drug are present as discrete parts (moieties) of a single continuous polypeptide chain.

Thus the methods of the disclosure may be applied to one or more of: a therapeutic protein, a monoclonal antibody (mAb), a domain antibody (dAb), a dAb conjugate, a dAb fusion, a mAbdAb, or any other antigen binding protein described above.

For example, the antigen binding protein is a peptide-dAb fusion (eg Exendin 4-AlbudAb™/Dat01), a dAb conjugate (eg AlbudAb™ with a C-terminal cysteine (for PYY chemical conjugation)/Dat06), a dAb-dAb fusion (eg AlbudAb™-TNFR1 VH dAb/DOM100), or a naked dAb (eg VH dAb (anti-TNFR1)/DOM101).

For example, the antigen binding protein comprises or consists of SEQ ID NO:1 (Dat01); SEQ ID NO:3 (Dat06); SEQ ID NO:5 (DOM100); SEQ ID NO:7 (DOM101); or SEQ ID NO:9 (DOM101 alanine-extended).

### Expression of Protein

Suitable microbial cells can be prokaryotic, including bacterial cells such as Gram negative or Gram positive bacteria. Such bacterial cells include Escherichia Coli (for example, strain W3110, or BL21), Bacilli sp., (for example B. subtilis), Pseudomonas sp., Moraxella sp., Corynebacterium sp., and other suitable bacteria.

Suitable microbial cells can be eukaryotic, including yeast (for example Saccharomyces cerevisiae, Pichia pastoris), or fungi (for example Aspergillus sp.).

A vector comprising a recombinant nucleic acid molecule encoding the recombinant protein is also described herein. The vector may be an expression vector comprising one or more expression control elements or sequences that are operably linked to the recombinant nucleic acid. Examples of vectors include plasmids and phagemids.

Suitable expression vectors can contain a number of components, for example, an origin of replication, a selectable marker gene, one or more expression control elements, such as a transcription control element (eg promoter, enhancer, terminator) and/or one or more translation signals, a signal sequence or leader sequence. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired cell. Promoters can be constitutive or inducible. For example, a promoter can be operably linked to a nucleic acid encoding an antibody, antibody chain or portion thereof, such that it directs transcription of the nucleic acid. A variety of suitable promoters for prokaryotic cells (e.g, lac, tac, trp, phoA, lambdapL, T3, T7 (T7A1, T7A2, T7A3) promoters for E. coli) may be used. Operator sequences which may be employed include lac, gal, deo and gin. One or more perfect palindrome operator sequences may be employed.

In addition, expression vectors typically comprise a selectable marker for selection of cells carrying the vector, and, in the case of a replicable expression vector, an origin of replication. Genes encoding products which confer antibiotic or drug resistance are common selectable markers and may be used in prokaryotic (eg lactamase gene (ampicillin resistance), Tet gene for tetracycline resistance) and eukaryotic cells (eg neomycin (G418 or geneticin), gpt (mycophenolic acid), ampicillin, or hygromycin resistance genes). Dihydrofolate reductase marker genes permit selection with methotrexate in a variety of cells.

An expression vector as described in WO2007/088371 (for example pAVE037, pAVE007, or pAVE011) may be used to express the protein. Alternatively, a commercially available vector such as pJExpress401 may be used to express the protein.

The host cell comprises the recombinant nucleic acid molecule or vector described above.

The cells of the microbial cell broth of the present invention express a recombinant protein. The recombinant protein may be expressed intracellularly. In another aspect, the expressed recombinant protein has a signal sequence (also known as a signal peptide), which routes the protein along the secretory pathway of the microbial cell.

In Gram-positive bacteria, secreted proteins are most commonly translocated across the single membrane by the Sec pathway or the Tat pathway. In Gram-negative bacteria, some secreted proteins are exported across the inner and outer membranes in a single step via the type I, type III, type IV or type VI secretion pathways, whereas other proteins are first exported into the periplasm via the universal Sec or Tat pathways and then translocated across the outer membrane mainly via the type II or type V machinery. The type II system involves a two-step process in which a premature protein containing a Sec secretion sequence is exported to the periplasm using the Sec pathway. The secretion sequence is removed by proteolysis resulting in a mature, processed protein being present in the periplasm and whether or not the protein is secreted to the culture medium highly depends on the characteristics of secretion sequence, protein, cell and culture conditions. Also in the case of cell lysis (autolysis) it can be assumed that the majority of the protein in the culture medium originates from the periplasm and therefore is processed. The recombinant protein may be actively secreted into the culture medium via the secretory signal sequence; or passively from the periplasm to the culture medium via other cellular pathways known in the art.

Processing of the signal sequence includes cleavage and removal of the signal sequence from the protein. However, some amino acids of the signal sequence are known to remain at the N-terminus of the protein, such that the signal sequence is not properly processed. The signal sequence may be 90% or more processed, such that 10% or less of the signal remains at the N-terminus of the protein. The signal sequence may be at least 91, 92, 93, 94, 95, 96, 97, 98, or 99% processed. The signal sequence may about 100% processed, such that none remains at the N-terminus of the protein following passage through the secretory pathway of the cell.

The signal sequence may be a periplasmic targeting signal sequence. Signal sequences to direct proteins to the periplasm are known in the art. For example, a MalE signal sequence is used. Alternatively, a PelB or OmpA signal sequence is used.

### Harvest

The microbial host cell is grown under suitable conditions to express the recombinant protein. A microbial cell broth is a population of host cells that express the recombinant protein. The microbial cell broth may be produced using fed batch fermentation of host cells (for example Escherichia coli) with media (such as complex media) in fermentation vessels following standard procedures. Fermentation conditions include feeding the cells with nutrients and an air supply.

Harvest is the end of fermentation. Harvest may be at any time point during fermentation that is considered sufficient to end the fermentation process and recover the recombinant protein being expressed. Harvest may occur between 8 and 50 hours post induction of the cell broth to express the recombinant protein. For example, harvest may occur between 8 and 36 hours post induction. At harvest, the solid content of the microbial cell population may be between 5-30% Wet Cell Weight (WCW).

The fermentor volume may be:

(i) about 10,000 litres; about 5,000 litres; about 2,000 litres; about 1,000 litres; about 500 litres; about 125 litres; about 50 litres; about 20 litres; about 10 litres; about 5 litres; or

(ii) between 5 and 10,000 litres; between 10 and 5,000 litres; between 20 and 2,000 litres; between 50 and 1,000 litres.

The particle size distribution of the harvest may be considerably variable, with greater or lesser extent of fine (≦35 μm) particle formation. For example, the percentage by total volume of particles ≦5 μm may be 5% or more, 10% or more, 25% or more, 50% or more, 75% or more, 80% or more, 85% or more, 90% or more, 95% or more, or 100%.

The harvest may comprise cells that have naturally lysed, also known as auto-lysis. For example, 1-50% of the cells in the harvest may have undergone autolysis. Alternatively, 20-50%; or 30-50%; or 40-50% of the cells in the harvest have autolysed. Alternatively, 10% or more; 20% or more; 30% or more; 40% or more; or 50% or more of the cells in the harvest have autolysed. Autolysis may be indirectly determined by DNA concentration in a clarified harvest, or by capacitance, as described in the Examples. Autolysis could also be indirectly determined by release/secretion of the recombinant protein into the culture medium, but this is not necessarily a direct correlation since there are other ways by which release/secretion into the medium could occur (as discussed above).

Harvest may include the optional step of emptying the fermentor of the microbial cell broth.

### Optional Pre-Treatment of Harvest

Pre-treatment of the harvest is a method of conditioning the harvest. This step may be carried out in the fermentor, or after the harvest has been removed from the fermentor. Pre-treatment includes: thermally, mechanically or chemically lysing the harvest (for example by homogenisation, freeze-thaw, lysis); and periplasmic extraction. At least one periplasmic extract may be extracted using methods known in the art. The protein may be expressed intracellularly, and the cells may be lysed to release the protein. For example, the cells may be homogenised to release the protein from inside the cell, or from within the periplasm.

In one embodiment, the harvest is not further treated prior to addition of a flocculant. For example, the harvest is not a lysate, ie it is not treated with a chemical lysis reagent. For example, the harvest is not a homogenate. For example, the harvest is not subjected to freeze-thaw.

### Addition of Flocculant

It was hypothesised by the inventors that an improved clarification step would involve use of a flocculant to achieve a low proportion (5% or less) of fine (≦5 μm or less) particles in the harvest. As such the particle size distribution was monitored before addition of flocculant, and with increasing levels of flocculant.

Flocculants include: mineral or vegetable hydrocolloids; anionic polyelectrolytes (for example polystyrene sulfonate, anionic polyacrylamide); cationic polyelectrolytes (for example polyethyleneimine (PEI), cationic polyacrylamide), natural polymers from microorganisms (for example Chitosan); and chemical flocculants, for example aluminium sulphate, synthetic and non-synthetic polymers, strong cationic and. Specific examples of flocculants include PEI (MW: 50 kDa to 100 kDa), Poly(diallyldimethylammonium chloride) (PDADMAC) (low molecular weight version MW: 100 kDa to 200 kDa; or high molecular weight version 400 kDa to 500 kDa), Acid precipitation, CaCl₂, Chitosan (MW: 110 kDa). In one embodiment, the flocculant is PEI (50 kDa to 100 kDa). In another embodiment, the flocculant is PDADMAC low molecular weight version MW: 100 kDa to 200 kDa. In a further embodiment, the flocculant is PDADMAC high molecular weight version 400 kDa to 500 kDa. In another embodiment, the flocculant is CaCl₂.

Flocculants cause the aggregation of insoluble or solid material, such that the soluble recombinant protein remains in solution. PEI may act both as a “precipitant” of soluble materials such as nucleic acids, lipids, colloidal protein (not the recombinant protein); and as a “flocculant” of cells and cell debris, such that the recombinant protein stays in solution.

An amount of the flocculant is added to the harvest to achieve a particle size distribution by volume of about 5% or less particles in the size range of 5 μm or less. This amount of flocculant may be between 0.01-5% by volume of the harvest. Alternatively, the amount of flocculant is between 0.01-2% by volume of the harvest. For example the amount of flocculant may be between 0.1 and 2%, between 0.1 and 0.5%; or between 0.3 and 0.5%, or is 0.5%, by volume of the harvest.

For example, the PEI, PDADMAC low molecular weight version (MW: 100 kDa to 200 kDa), or PDADMAC high molecular weight version (400 kDa to 500 kDa), is at a concentration of between 0.1 to 2%. Alternatively, the CaCl₂ is at a concentration of between 3 to 6%, for example at 4.3%. For example, the PEI concentration in the DOM100 harvest is 0.1-2.0%, 0.15-2.0%, 0.2-2.0%, or 0.3-0.5%. Alternatively, the CaCl₂ concentration in the DOM100 harvest is 4.3%. For example, the PEI concentration in the Dat01 harvest is between 0.05-0.8%, 0.1-0.8%, or 0.1-0.2%. For example, the PEI concentration or PDADMAC (high or low) concentration in the Dat06 harvest is between 0.1-0.5%, 0.2-0.5%, or 0.15-0.4%. For example, the PEI concentration in the DOM101 harvest is 0.5%.

The particle size distribution of the flocculated harvest should be about 5% or less particles in the size range of 5 μm or less. This is independent of the starting proportion of particles in the size range of 5 μm or less of the harvest pre-flocculant addition. Thus, if the percentage of particles in the size range of 5 μm or less in the harvest is higher than 5%, then addition of flocculant should reduce this percentage to about 5% or below. If the percentage of particles in the size range of 5 μm or less in the harvest is about 5% or below, then addition of flocculant should maintain this percentage to about 5% or below.

The time elapsed between the harvesting step and the addition of flocculant may be between 0 to 24 hours. Alternatively, the time elapsed between the harvesting step and the addition of flocculant may be between 0 to 12 hours, 0 to 6 hours, or 0 to 3 hours.

Particle size distributions may be determined using a Malvern Master Size Instrument equipped with a Small Volume Dispersion Unit (Malvern instruments, Worcestershire, UK) according to manufacturer's recommended protocols.

The refractive index (RI) may be set between 1.4 to 1.6. For example, the RI may be set at 1.45, or 1.52, or 1.59. The adsorption coefficient may be set between 0.000 and 0.001. For example the adsorption coefficient may be set at 0.000 or 0.001.

The percentage of particles in the size distribution of 5 μm may be about 5%, or less; about 4%, or less; about 3%, or less; about 2.5%, or less; about 2%, or less; about 1.5%, or less; about 1%, or less; about 0.5%, or less; about 0.25, or less; about 0.1%, or less; about 0.05%, or less; about 0.01%, or less; or about 0%, following addition of the flocculant.

For example, the percentage of particles in the size distribution of 5 μm is in the range of 0-6%, 0-5%, 0-4%, 0-3%, 0-2.5%, 0-2%, 0-1.5%, 0-1%, 0-0.05%, or 0-0.01%.

The size range of particles in the 5 μm or less volume may be about 4 μm, or less; about 3 μm, or less; about 2.5 μm, or less; about 2 μm, or less; about 1.5 μm, or less; about 1 μm, or less; about 0.5 μm, or less. For example, the size range may be from 0-5 μm, 0-4 μm, 0-3 μm, 0-2 μm, or 0-1 μm.

A first amount of a flocculant may be added, the particle size distribution assessed, and if necessary, a second amount of a flocculant added to achieve a particle size distribution by volume of about 5% or less particles in the size range of 5 μm or less.

### Clarification

Clarification is the process to remove solid particulates. Clarification can lower the burden on subsequent chromatographic steps during purification. Typical clarification steps comprise a settling step—also known as sedimentation (eg by gravity), and/or a centrifugation step, and/or a filtration step.

The centrifugation step may be continuous centrifugation (eg. with a continuous feed zone). The centrifuge may in itself be operating “batch” or “intermittently” or “continuously” with respect to discharging the solids. For example, a tubular bowl centrifuge may be used as the continuous centrifugation step.

The percentage solids remaining after centrifugation may be about 0%; about 0.5%, or less; about 1%, or less; about 2%, or less; about 3%, or less; about 4%, or less; about 5%, or less; about 10%, or less; about 15%, or less; or about 20%, or less.

Centrifugation may be used as the sole clarification process. Alternatively, centrifugation may be used in combination with filtration to provide a combined clarification process. Centrifugation may occur as the first step and then filtration as a subsequent step, or visa versa. Alternatively, filtration may be used as the sole clarification process. Filtration (for example depth filtration) can provide further clarification, removing small solid particles.

The filter capacity may be improved by about 200%; about 300%, or more; about 400%, or more; about 500%, or more; about 600%, or more; about 700%, or more; about 800%, or more; about 900%, or more; about 1000%, or more; or about 2000%, or more, with the addition of flocculant compared with no flocculant.

### Purification of the Recombinant Protein

Clarification is often followed by purification to ensure adequate purity of the recombinant protein. One or more chromatography steps may be used, for example one or more chromatography resins; and/or one or more filtration steps. For example affinity chromatography using resins such as protein A or L may be used to purify the recombinant protein. Alternatively, or in addition to, an ion-exchange resin such as a cation-exchange may be used to purify the recombinant protein.

### Recombinant Protein Recovery

Four different recombinant proteins are described in the Examples. There is no indication that protein recovery is impaired by the use of flocculant as described by the methods herein. It may be possible that use of flocculant as described by the methods herein actually improves protein release from the cell.

### Other Factors

Altering the pH of the harvest upon addition of a flocculant may be used to fine tune the number of particles 5 μm and below. For example, the pH of the harvest plus flocculant may be adjusted to pH≦7. The pH of the harvest plus flocculant may be adjusted to pH4-7; or pH4-6; or pH4-5.

Altering the conductivity of the harvest upon addition of a flocculant may be used to fine tune the number of particles 5 μm and below, or the mean particle diameter.

The following items describe the present invention:

Item 1. A method of producing a recombinant protein, wherein the method comprises:
(a) harvesting a microbial cell broth that expresses the recombinant protein; and
(b) adding an amount of a flocculant to achieve a particle size distribution by volume of about 5% or less particles in the size range of 5 μm or less.
   Item 2. The method of item 1, wherein the method further comprises step:
(c) clarifying the flocculated harvest.
   Item 3. The method of item 2, wherein the method further comprises step:
(d) purifying the recombinant protein from the clarified flocculated harvest.
   Item 4. A method of clarifying a microbial harvest, wherein the method comprises:
(a) harvesting a microbial cell broth;
(b) adding an amount of a flocculant to achieve a particle size distribution by volume of about 5% or less particles in the size range of 5 μm or less; and
(c) clarifying the flocculated harvest.
   Item 5. The method of item 4, wherein the microbial cell broth expresses a recombinant protein.
   Item 6. The method of any one of the preceding items, wherein the time elapsed between the harvesting step of (a) and the flocculant addition in step (b) is between 0 to 24 hours.
   Item 7. The method of any one of the preceding items, wherein the method further comprises an additional step between step (a) and (b):
(b′) pre-treating the harvest by (i) mechanical or chemical lysis, or (ii) periplasmic extraction.
   Item 8. The method of any one of items 1 to 6, wherein the harvested microbial cell broth of step (a) is not further treated prior to step (b).
   Item 9. The method of any one of items 2 to 8, wherein step (c) comprises (i) settling; and/or (ii) centrifugation; and/or (iii) filtration.
   Item 10. The method of any one of items 1 to 3 and 5 to 9, wherein the expressed recombinant protein comprises a signal sequence.
   Item 11. The method of item 10, wherein the signal sequence of the secreted recombinant protein is more than 90% processed.
   Item 12. The method of item 10 or 11, wherein the signal sequence is a periplasmic targeting signal sequence.
   Item 13. The method of any one of items 1 to 3 and 5 to 12, wherein the recombinant protein is secreted into the culture medium.
   Item 14. The method of any one of the preceding items, wherein 1-50% of the cells in the microbial cell broth of (a) have undergone autolysis.
   Item 15. The method of item 14, wherein autolysis is assessed by capacitance.
   Item 16. The method any one of the preceding items, wherein the method further comprises in step (b) adding a first amount of a flocculant, assessing the particle size distribution, and if necessary, adding a second amount of a flocculant to achieve a particle size distribution by volume of about 5% or less particles in the size range of 5 μm or less.
   Item 17. The method of any one of the preceding items, wherein the amount of the flocculant is added in an amount of between 0.01-5% by the volume of the harvest.
   Item 18. The method of any one of the preceding items, wherein the amount of the flocculant is added in an amount of between 0.01-2% by the volume of the harvest.
   Item 19. The method of item 18, wherein the flocculant is polyethylenimine (PEI), or poly(diallyldimethylammonium chloride) (PDADMAC).
   Item 20. The method of item 19, wherein the PEI is high molecular weight PEI, for example MW 50 kDa-100 kDa.
   Item 21. The method of item 18, wherein the flocculant is CaCl₂.
   Item 22. The method of any one of the preceding items, wherein the microbial cell broth is an Escherichia coli cell broth.
   Item 23. The method of any one of the preceding items, wherein the % of particles in the size distribution of 5 μm is about 4%, or less; about 3%, or less; about 2.5%, or less; about 2%, or less; about 1.5%, or less; about 1%, or less; about 0.5%, or less; about 0.25, or less; about 0.1%, or less; about 0.05%, or less; about 0.01%, or less; or about 0%, following addition of the flocculant in step (b).
   Item 24. The method of any one of the preceding items, wherein the size range of particles in the 5 μm or less volume is: about 4 μm, or less; about 3 μm, or less; about 2.5 μm, or less; about 2 μm, or less; about 1.5 μm, or less; about 1 μm, or less; about 0.5 μm, or less.
   Item 25. The method of any one of items 9 to 24, wherein the centrifugation is by continuous centrifugation.
   Item 26. The method of any one of items 9 to 24, wherein the centrifugation is by batch centrifugation.
   Item 27. The method of any one of items 2 to 26, wherein the % solids remaining during step (c) is about 0%; about 0.5%, or less; about 1%, or less; about 2%, or less; about 3%, or less; about 4%, or less; about 5%, or less; about 10%, or less; about 15%, or less; or about 20%, or less.
   Item 28. The method of any one of items 2 to 27, wherein the filter capacity during step (c) is improved by about 200%; about 300%, or more; about 400%, or more; about 500%, or more; about 600%, or more; about 700%, or more; about 800%, or more; about 900%, or more; about 1000%, or more; or about 2000%, or more, in the presence of flocculant compared with no flocculant.
   Item 29. The method of any one of items 1 to 3, and 5 to 28, wherein the recombinant protein is an antigen binding protein.
   Item 30. The method of item 29, where the antigen binding protein comprises a dAb (domain antibody).
   Item 31. The method of item 29 wherein the antigen binding protein comprises:
(a) a peptide-dAb fusion;
(b) a dAb conjugate;
(c) a dAb-dAb fusion; or
(d) a naked dAb.
   Item 32. The method of item 29 wherein the antigen binding protein comprises:
(a) Exendin 4-AlbudAb™ (SEQ ID NO:1);
(b) AlbudAb™ with a C-terminal cysteine (SEQ ID NO:3);
(c) AlbudAb™-TNFR1 VH dAb (SEQ ID NO:5); or
(d) VH dAb anti-TNFR1 (SEQ ID NO:7 or 9).
   Item 33. The method of any one of items 1 to 3, and 5 to 28, wherein the recombinant protein comprises a viral protein, a bacterial toxin, a bacterial toxoid, or a cancer antigen.
   Item 34. The method of any one of the preceding items, wherein the solid content of the harvest in (a) is 5-30% Wet Cell Weight (WCW).
   Item 35. The method of any one of the preceding items, wherein the microbial cell broth is harvested from a fermentor.
   Item 36. The method of item 35, wherein the fermentor volume is:
(i) about 10,000 litres; about 5,000 litres; about 2,000 litres; about 1,000 litres; about 500 litres; about 125 litres; about 50 litres; about 20 litres; about 10 litres; about 5 litres; or
(ii) between 5 and 10,000 litres; between 10 and 5,000 litres; between 20 and 2,000 litres; between 50 and 1,000 litres.
   Item 37. A modified Escherichia coli cell harvest wherein:
(a) the cells express a periplasmic targeted recombinant protein;
(b) the harvest comprises 0.01-2% PEI by volume; and
(c) the particle size distribution by volume of the harvest is about 5% or less particles in the size range of 5 μm or less.
   Item 38. The modified harvest of item 37, wherein the harvest has been treated by (i) mechanical or chemical lysis, or (ii) periplasmic extraction.
   Item 39. The modified harvest of item 37 or 38, wherein 1-50% of the cells have undergone autolysis.
   Item 40. The modified harvest of item 39, wherein autolysis is assessed by capacitance.
   Item 41. The modified harvest of any one of items 37 to 40, wherein the polyethylenimine (PEI) is high molecular weight PEI, for example MW 50 kDa-100 kDa.
   Item 42. The modified harvest of any one of items 37 to 41, wherein the % of particles in the size distribution of 5 μm is about 4%, or less; about 3%, or less; about 2.5%, or less; about 2%, or less; about 1.5%, or less; about 1%, or less; about 0.5%, or less; about 0.25, or less; about 0.1%, or less; about 0.05%, or less; about 0.01%, or less; or about 0%.
   Item 43. The modified harvest of any one of items 37 to 42, wherein the size range of particles in the 5 μm or less volume is about 4 μm, or less; about 3 μm, or less; about 2.5 μm, or less; about 2 μm, or less; about 1.5 μm, or less; about 1 μm, or less; about 0.5 μm, or less.
   Item 44. The modified harvest of any one of items 37 to 43, wherein the recombinant protein comprises an antigen binding protein.
   Item 45. The modified harvest of item 44, where the antigen binding protein comprises a dAb (domain antibody).
   Item 46. The modified harvest of item 44, wherein the antigen binding protein comprises:
(a) a peptide-dAb fusion;
(b) a dAb conjugate;
(c) a dAb-dAb fusion; or
(d) a naked dAb.
   Item 47. The modified harvest of item 44, wherein the antigen binding protein comprises:
(a) Exendin 4-AlbudAb™;
(b) AlbudAb™ with a C-terminal cysteine;
(c) AlbudAb™-TNFR1 VH dAb; or
(d) VH dAb anti-TNFR1.
   Item 48. The modified harvest of any one of items 37 to 43, wherein the recombinant protein comprises a viral protein, a bacterial toxin, a bacterial toxoid, or a cancer antigen.
   Item 49. The modified harvest of any one of items 37 to 48, wherein the solid content of the harvest is 5-30% Wet Cell Weight (WCW).
   Item 50. The modified harvest of any one of items 37 to 49, wherein the harvest volume is:
(i) about 10,000 litres; about 5,000 litres; about 2,000 litres; about 1,000 litres; about 500 litres; about 125 litres; about 50 litres; about 20 litres; about 10 litres; about 5 litres; or
(ii) between 5 and 10,000 litres; between 10 and 5,000 litres; between 20 and 2,000 litres; between 50 and 1,000 litres.

### EXAMPLES

All chemicals and reagents used are from Sigma Aldrich unless otherwise stated.

The flocculant Polyethyleneimine (PEI) is a cationic polymer comprised of primary, secondary and tertiary amines, (C2H5N)ₙ, MW=50,000-100,000 Da and was prepared as a 10% or 12.5% w/v solution in water and aged for at least 30 minutes prior to use.

The flocculant Poly(diallyldimethylammonium chloride) (PDADMAC) is a high charge density cationic polymer used at either the low molecular weight version (100,000-200,000 Da) or the high molecular weight version (400,000-500,000 Da).

Four exemplar recombinant proteins are used in the examples and they are described below in Table 1.

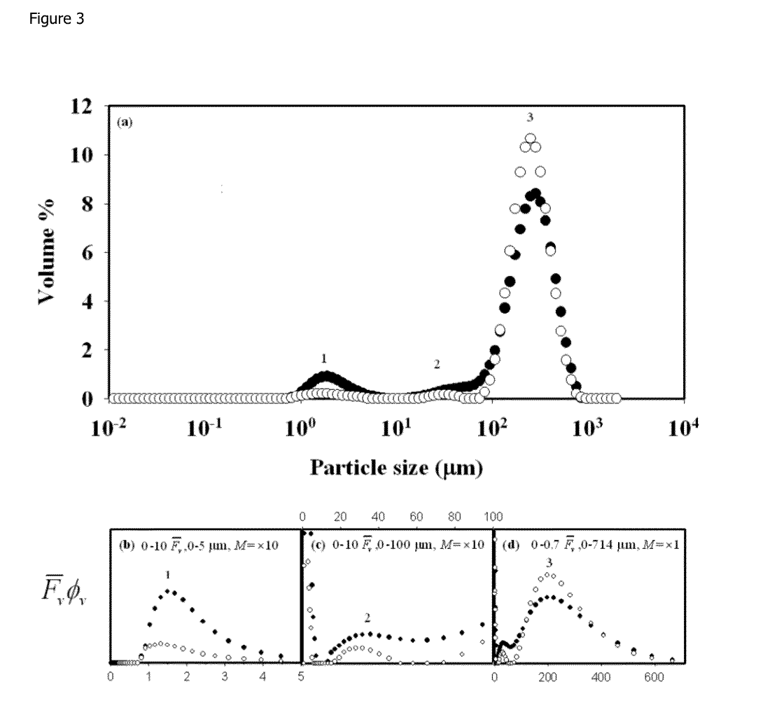

TABLE 1

| | | | | | Patent |
|---|---|---|---|---|---|
| | | | | | application no. |
| | | E. coli | | Signal | describing the |
| Recombinant Protein | | host cell | Vector | sequence | protein |
| Exendin 4 - | Dat01/ | W3310 | pAVE037 | MalE | WO2010108937 |
| AlbudAb ™ | DMS7139/ | | | | SEQ ID NO: 24 |
| SEQ ID NO: 1 | Exendin 4, (G4S)3, | | | | |
| | linker Dom7h-14-10 | | | | |
| | fusion | | | | |
| AlbudAb ™ with C- | Dat06/ | W3310 | pJ | MalE | WO2011039096 |
| terminal cysteine - | Dom7h-11-15 | | Express401 | | SEQ ID NO: 47 |
| for PYY chemical | (R108C) | | | | |
| conjugation | | | | | |
| SEQ ID NO: 3 | | | | | |
| AlbudAb ™ - TNFR1 | DOM100/Dom0100 - | W3310 | pAVE007 | PelB | WO2011051217 |
| VH dAb, fusion | DMS5541/Dom1h- | | | | SEQ ID NO: 66 |
| SEQ ID NO: 5 | 574-208); | | | | |
| | and-Dom7h-11-3 | | | | |
| VH dAb (anti- | DOM101/Dom0101/ | W3110 | pAVE011 | OmpA | WO2008149148 |
| TNFR1) | Dom1h-131-206 | | | | FIG. 3 |
| SEQ ID NO: 7 | | | | | |

The work carried out here with DOM101 (SEQ ID NO:7) is thought to be directly equivalent to the results predicted for alanine-extended DOM101 (SEQ ID NO:9).

Proteins were produced using fed batch fermentation of Escherichia coli with complex media in 1 L fermentation vessels following standard procedures. Fermentations were then harvested under appropriate conditions between 8 and 50 hours post induction.

Particle size distributions were determined using a Malvern Mastersize Instrument equipped with a Small Volume Dispersion Unit (Malvern instruments, Worcestershire, UK) according to manufacturer's recommended protocols. The Refractive index (RI) ranged from 1.4 to 1.6. The adsorption coefficient ranged from 0 to 0.001.

### Example 1

Three proteins were used in this study. Dom100, Dat06 and Dat01 are all recombinant proteins that comprise a domain antibody (dAb) as described in Table 1.

The pre-prepared 10% PEI solution was added to the fermentation harvest to give the desired concentration for study. This was then mixed for 1 hour at room temperature prior to particle size distribution measurement.

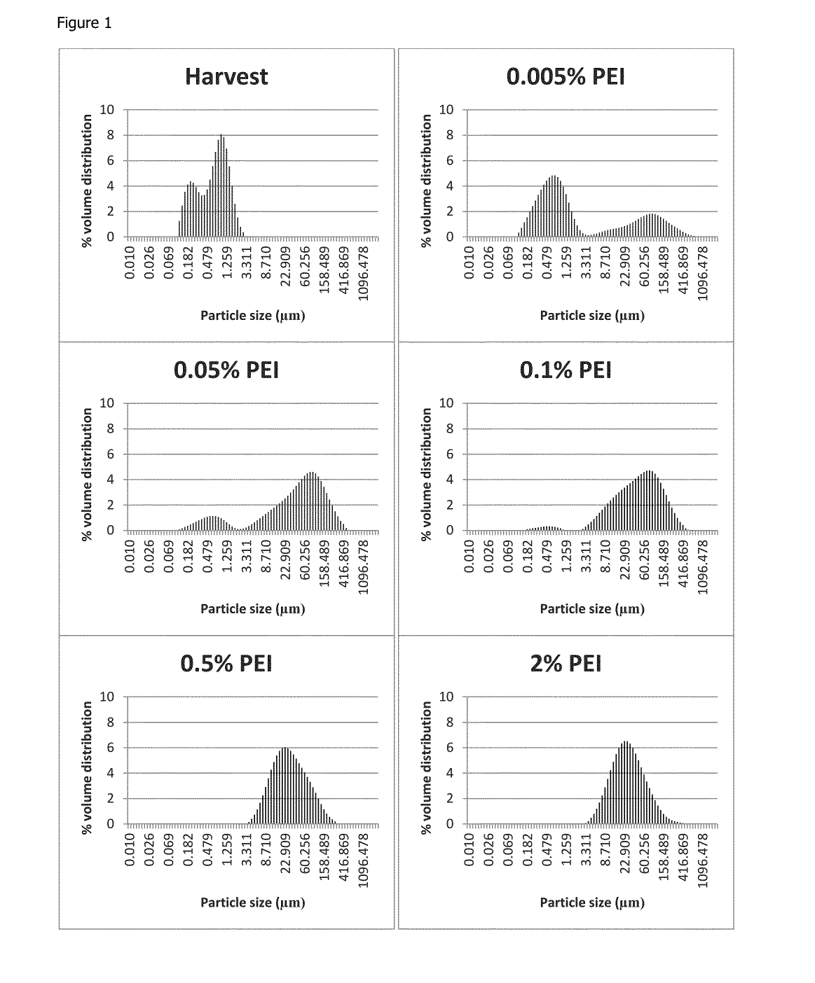

The particle size distribution is given for the DOM100 harvest and with the addition of 0.005%, 0.05%, 0.1%, 0.5% and 2% PEI in FIG. 1. The harvest (with no addition of flocculant) can be seen to comprise a majority of particles by volume ≦5 μm in diameter. However, it is important to note that separate studies (not shown here) indicate that there can be considerable variability in the particle size distribution of the harvest, with greater or lesser extent of particles by volume ≦5 μm in diameter. FIG. 1 shows that by increasing the amount of PEI, the presence of ≦5 μm particles in the distribution is reduced. At 0.5% PEI the large majority of particles ≦5 μm in diameter have been removed.

A more detailed account of this shift in particle size distribution of harvest expressing DOM100 upon addition of PEI is shown in Table 2 along with the data for the harvests expressing Dat06 or Dat01. The focus of Table 2 is not on the larger particles/aggregates that are often the focus of studies with flocculant, but instead on the percentage of particles that are ≦5 μm, by total volume of the harvest, or flocculated harvest.

TABLE 2
Percentage volume of particles ≦5 μm in diameter with increasing
levels of PEI for harvests expressing DOM100, Dat06 or Dat01.

| | % volume of particles ≦5 μm | | |
|---|---|---|---|
| | diameter by total volume | | |
| % PEI | Dat06 | Dom0100 | Dat01 |
| 0 | 100 | 100 | 97.04 |
| 0.005 | | 66.02 | |
| 0.01 | 57.10 | 51.97 | |
| 0.025 | | 27.81 | |
| 0.03 | 28.93 | | |
| 0.05 | 24.27 | 16.25 | 5.09 |
| 0.075 | | 9.69 | |
| 0.1 | 6.15 | 4.37 | 1.56 |
| 0.150 | | 2.35 | |
| 0.2 | | 1.63 | 0.83 |
| 0.3 | | 1.31 | |
| 0.4 | | | 4.02 |
| 0.5 | 5.35 | 1.34 | |
| 0.8 | | | 2.41 |
| 2.0 | 17.93 | 1.60 | |

For the DOM100 harvest, the proportion of ≦5 μm particles is reduced upon the addition of PEI. In particular, the PEI concentration that achieves a particle size distribution by volume of about 5% or less of particles ≦5 μm is between 0.1%-2.0% (upper limit tested). The optimal sweet spot seems to be at the concentration of 0.2-2.0% (less than 2% by volume), or at 0.3-0.5% (less than 1.5% by volume).

For the Dat01 harvest, the PEI concentration that achieves a particle size distribution by volume of about 5% or less of particles ≦5 μm is between 0.1%-0.8% (upper limit tested). The optimal sweet spot seems to be at the concentration of 0.1-0.2% (less than 1.6% by volume).

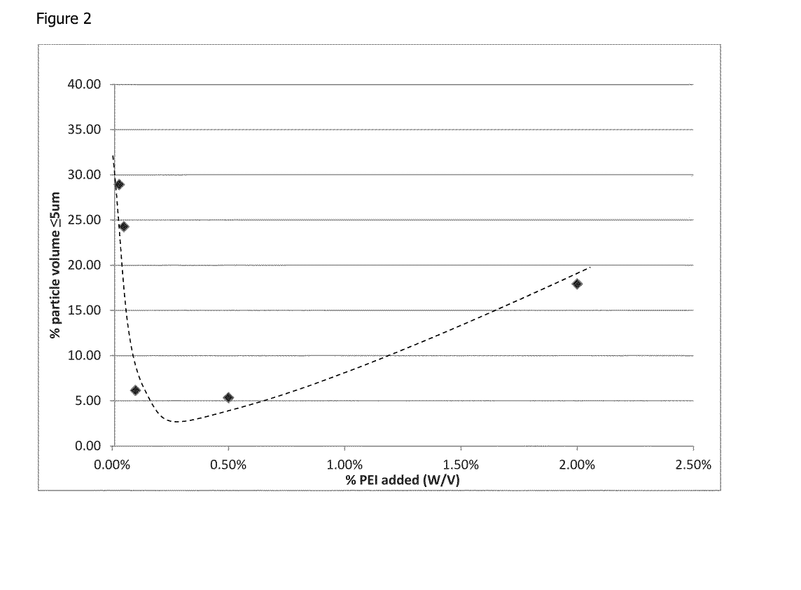

For the Dat06 harvest, the PEI concentration that achieves a particle size distribution by volume of about 5% or less of particles ≦5 μm is between 0.1%-0.5%. Note that for this harvest, “about 5%” is equal to 6.15% and 5.35%. It is postulated that Dat06 harvest particle size distribution would reduce to below 5% in the range 0.1-0.5% PEI and this is demonstrated in FIG. 2. The data (except for 0% PEI (100%) and 0.01% PEI (57%)) described in Table 2 is plotted in FIG. 2 for Dat06 harvest with an extrapolated line to demonstrate the hypothesis that the % volume distribution should drop below 5% of ≦5 μm particles between the experimentally derived points of 0.1%-0.5% PEI. Thus the predicted optimal sweet spot would be 0.15-0.4% PEI for this Dat06 harvest. Two other Dat06 harvests were analysed: harvest A contained a metal chelator (EDTA), and harvest B was controlled during fermentation to have a low cell mass. With no addition of PEI, the % volume of particles ≦5 μm diameter by total volume was 97.09% for harvest A; and 93.78% for harvest B. These percentages were reduced to about ≦5% of ≦5 μm particles at PEI concentrations of 0.1%-0.4% for harvest A (1.79%-5.62% ≦5 μm particles); and 0.1%-0.5% PEI for harvest B (0.64%-1.73% ≦5 μm particles). These were not analysed further.

Thus, it can be seen that increasing the amount of flocculant does not directly correspond with a reduced percentage of particles in the ≦5 μm range. An optimum amount of flocculant can be identified, and this optimum amount has improved effects as shown below.

### Example 2

A fourth example recombinant protein was used in this study. DOM101 is described in Table 1. Particle size distributions for harvests expressing DOM101 were calculated as described above.

The impact of shear is investigated in the present study since typically shear conditions exhibited at the lab-scale are substantially less than those exhibited at large manufacturing scale. As such the impact of shear is often ignored or under-estimated in early process research conducted at a lab-scale.

Two different levels of shear were studied: “low shear” equivalent maximum power dissipation εmax, of 0.04×10⁶ W kg⁻¹, and “high shear” equivalent maximum power dissipation εmax, of 0.53×10⁶ W kg⁻¹.

Appropriate samples were exposed to shear for 20 s in a rotary disc device (20 mL stainless steel chamber of 50 mm internal diameter and 10 mm height, fitted with a stainless steel rotating disc of 40 mm diameter and 1 mm thickness with disk speed (0-20,000 rpm) controlled by a custom designed power pack (UCL mechanical workshop, UCL, London, see also McCoy R, Hoare M, Ward S. 2009. Ultra scale-down studies of the effect of shear on cell quality; Processing of a human cell line for cancer vaccine therapy. Biotechnology Progress 25(5):1448-1458.). The disc speed was related to maximum energy dissipation rates using a computational fluid dynamics derived correlation (for methodology involved, see for example Boychyn M, Doyle W, Bulmer M, More J, Hoare M. 2000. Laboratory scaledown of protein purification processes involving fractional precipitation and centrifugal recovery, Biotechnology and Bioengineering 69:1-10, now redefined and condensed into an empirical relationship ε=(1.7×10̂-3) (N̂3.71), where ε has units of W kg⁻¹ and N is speed in revs. sec-1, 100<N<200; and Chatel, A., Kumpalume, P. and Hoare, M. (2013), Ultra scale-down characterization of the impact of conditioning methods for harvested cell broths on clarification by continuous centrifugation—Recovery of domain antibodies from rec E. coli. Biotechnol. Bioeng. doi: 10.1002/bit.25164).

Particle size distributions are presented in FIG. 3 for harvest (open circle), and harvest exposed to high shear (closed circle). The size distributions are presented as (a) the total volume particle size distribution on logarithmic size scale, and the particle size distributions emphasizing peaks 1, 2 and 3, in inserts: (b), (c) and (d) respectively. The relative volume fractions, φv, is 0.11 for harvest and for sheared material. Axis scales for v F and d and the relative magnification, M, of the Figure are given in the inserts (b), (c) and (d). Volume ratio of peaks 1, 2, and 3 are 2:1:97 for harvest and 8:4:88 for sheared harvest. The particle size distribution observed is different to the three recombinant protein expressing harvests of Example 1, with a larger proportion of larger particles that are above 5 μm. As discussed above, separate studies, not shown here, indicate considerable variability in the size distribution of the harvest, with greater or lesser extent of fine particle formation.

Table 3 below shows the percentage of particles that are ≦5 μm, by total volume of the harvest, for each of the samples described above. As can be seen upon increased levels of shear associated with bioprocessing, particles in the ≦5 μm range increased in prevalence, such that more than 5% of the volume contains particles ≦5 μm. This would increase the burden on the subsequent clarification and purification steps.

### Addition of Flocculant

DOM101 harvest described above was subjected to PEI treatment as described in Example 1 to a final concentration of 0.5% w/v. Previous work (not shown here) on DOM101 harvest has already shown that 0.5% is the optimum amount of PEI. PEI-treated harvest was then subjected to shear as described above.

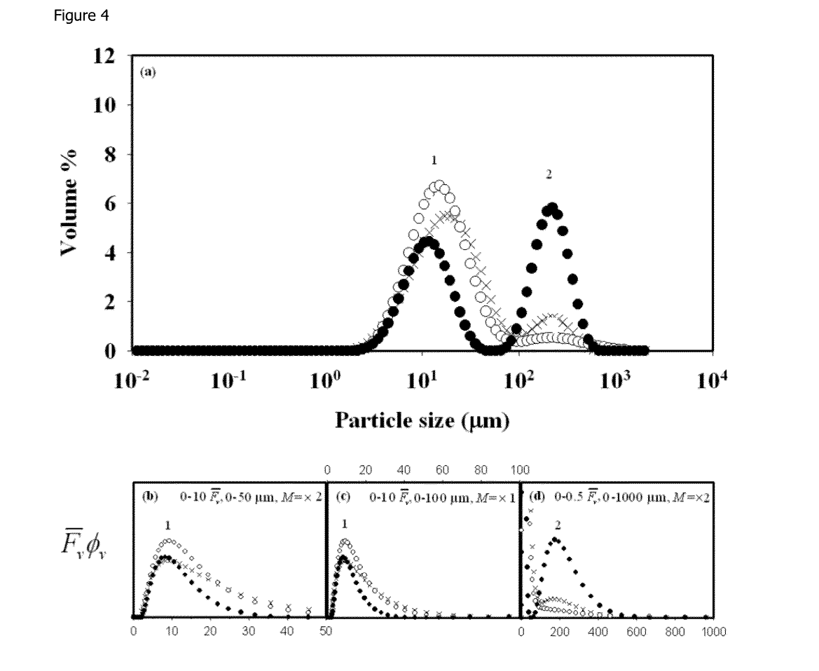

Particle size distributions are presented in FIG. 4 for PEI flocculated harvest (closed circle), and for PEI flocculated harvest sheared at low shear (cross) and high shear (open circle). The size distributions are presented as (a) the total volume particle size distribution on logarithmic size scale, and the particle size distributions emphasizing peaks 1, 1, and 2, in inserts: (b), (c) and (d) respectively. The volume ratios of peaks 1 and 2 are (PEI flocculated harvest) 50:50, (PEI flocculated low shear) 87:13, (PEI flocculated high shear) 93:7.

As can be seen the presence of PEI increases shifts the smallest particle size peak to a larger diameter point when compared to the non-PEI distribution in FIG. 3. This in turn however has minimal effect on the volume of ≦5 μm particles.

Table 3 below shows the percentage of particles that are ≦5 μm, by total volume of the harvest, for each of the samples described above. The particle size distribution by volume of about 5% or less of particles ≦5 μm in the presence of 0.5% PEI stays relatively constant in the presence of low and high shear. However, the percentage of particles ≦5 μm increases in the presence of high shear without the addition of PEI, to a further 6% of the total volume that is ≦5 μm. This data suggests that 0.5% PEI results in a more efficient and robust clarification step in the presence of shear.

TABLE 3
Percentage volume of particles ≦5 μm in diameter with increasing
shear for harvests expressing DOM10.

| | % volume of particles ≦5 μm | | |
|---|---|---|---|
| | diameter by total volume | | |
| Sample | No shear | Low shear | High shear |
| DOM101 | 2.02 | | 8.08 |
| DOM101 with | 4.44 | 5.83 | 5.66 |
| 0.5% PEI | | | |

### Example 3

DOM100 harvest was treated with PEI as described in Example 1 to the desired concentration. Samples were subjected to continuous centrifugation using a Carr Powerfuge at speed of 0.5 litres per minute (lpm) and 15325 revolutions per minute (rpm). The turbidity of the samples was then measured prior to centrifugation (feed turbidity) and after centrifugation (centrate turbidity) using standard conditions with a Hach turbidity meter (Colorado, US).

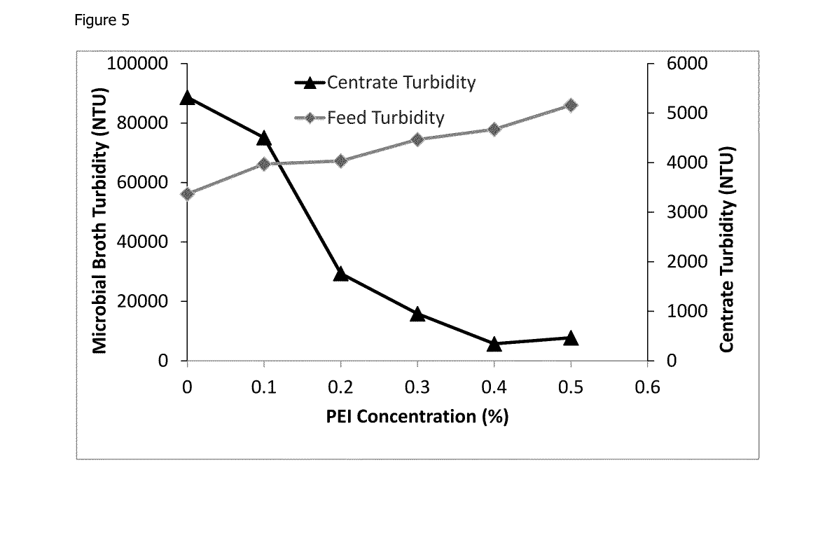

FIG. 5 demonstrates the effect on turbidity of increasing concentration of PEI addition to harvest pre and post centrifugation. The turbidity of the harvest pre-centrifugation (feed turbidity) shows a steady increase with addition of PEI consistent with the formation of floc. Centrate turbidity shows a decrease with increased levels of PEI consistent with a more efficient centrifugation process step. Centrate turbidity is measured on the right hand axis, and the feed turbidity is plotted on the left hand axis, because the centrate turbidity was orders of magnitude lower than that of feed turbidity. This improvement in turbidity post-centrifugation coincides with the 5% or lower ≦5 μm particles observed at the PEI concentrations of 0.1%-2.0% for DOM100 as shown in Table 2. In particular, the centrate turbidity improvement starts from 0.1% PEI, and improves up to the end point of 0.5% PEI in this study, with the optimum being at 0.4%. This coincides with the optimal sweet spot at the PEI concentration of 0.3-0.5% shown in Table 2 for DOM100 harvest.

### Example 4

DOM101 harvest was prepared as in Example 2 with and without PEI. Samples were then subjected to ultra-scale down centrifugation methodology using a method previously described by Tait A S, Aucamp J P, Bugeon A, Hoare M. 2009. Ultra scale-down prediction using microwell technology of the industrial scale clarification characteristics by centrifugation of mammalian cell broths. Biotechnology and Bioengineering 104(2):321-331. Percentage solids remaining were calculated by determining the relative decrease in optical density at an absorbance of wavelength 600 nm.

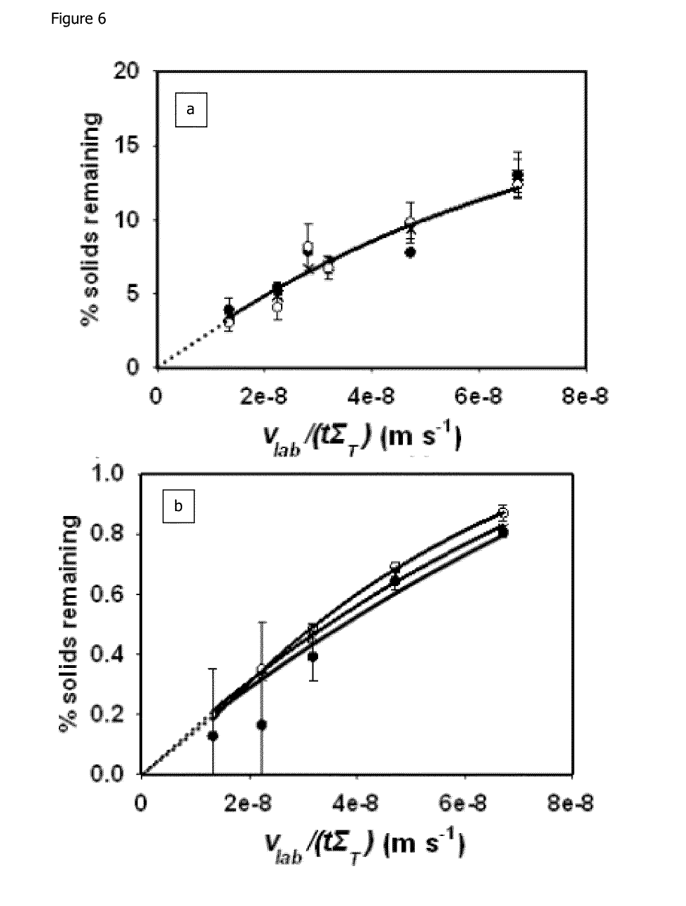

FIG. 6 demonstrates the % solids remaining for DOM101 harvest (a) and DOM101 harvest in the presence of 0.5% PEI (b). Also represented in each Figure is the sample subjected to no shear (closed circle), low shear (cross) and high shear (open circle) (shear is as described above in Example 2).

Data is presented as mean±s.d.; lines are best least square fit using 3rd order polynomials. For graph (a) single correlations are given as there is no consistent trend with increasing shear rate. In all cases the correlations are fitted through the origin which provides the control.

As can be seen in FIG. 6, the presence of 0.5% PEI significantly reduces the % solids remaining post centrifugation—the percentage solids remaining without PEI addition are present up to 10-15%, whereas with 0.5% PEI this is reduced to 0.8% solids remaining.

### Example 5

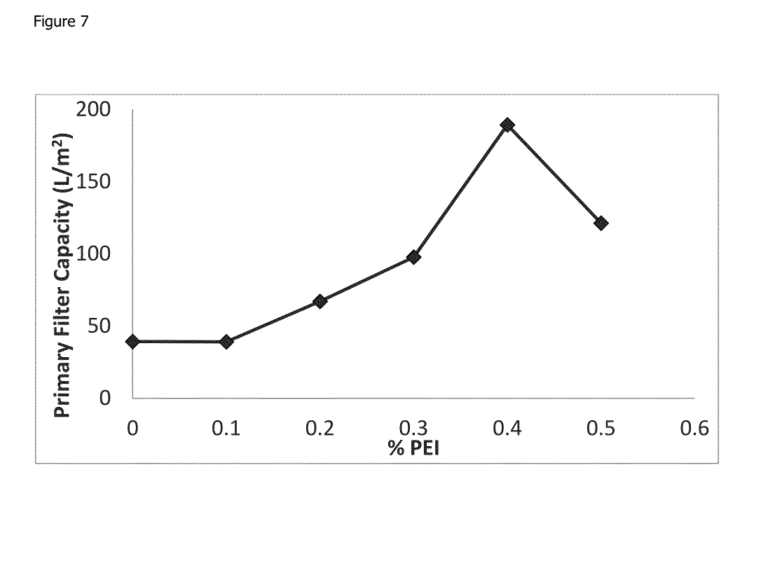

DOM100 harvest was prepared as in Example 1 with a range of PEI concentrations. This material was then passed through a centrifuge as described in Example 3, and then passed through a filter train comprising a primary and secondary filter. The maximum capacity of the primary filter (also known as Vₘₐₓ) prior to over-pressuring was calculated (L/m²) and plotted against % PEI added. As can be seen in FIG. 7, primary filter capacity rises substantially with increasing concentration of PEI, corresponding to the reduced presence of ≦5 μm particles in the harvest after flocculant addition. An improvement in filter capacity from the addition of PEI can be observed to start from 0.1% PEI and peaks at 0.4%, with an improvement still observed at the end-point of 0.5% in this study. The optimum appears to be at 0.4% PEI. This, together with Example 3 and Table 2 demonstrates the significant improvement in clarification of DOM100 harvest with a level of flocculant that achieves 5% or lower of the total particles in the range ≦5 μm. This improvement coincides with the 5% or lower ≦5 μm particles observed at the PEI concentrations of 0.1%-2.0% for DOM100 as shown in Table 2, and in particular, the optimal sweet spot at the PEI concentration of 0.3-0.5% shown in Table 2 for DOM100 harvest.

### Example 6

Dat06 and DOM100 harvests were treated as described below. Control harvests were clarified by centrifugation and DNA levels were measured with the Quant-iT dsDNA Broad Range Assay kit from Invitrogen according to manufacturer's instructions. All other harvests were homogenised using a Gaulin-type homogenized at a target pressure of 10,000 psi for 2 passes. These homogenised harvests were treated with increasing concentrations of either PEI (for Dat06 and DOM100 harvests) or high or low MW PDADMAC (for Dat06 harvests) and then clarified by centrifugation. DNA levels were measured as described above for the control harvests.

DNA can be considered to be an indicator of cell lysis—in the presence of intact cells there should be very little present in the supernatant. Presence of DNA is likely in itself to affect clarification as it increases the viscosity of the supernatant and can contribute to loss in effective centrifuge clarification and reduced filter flux rates.

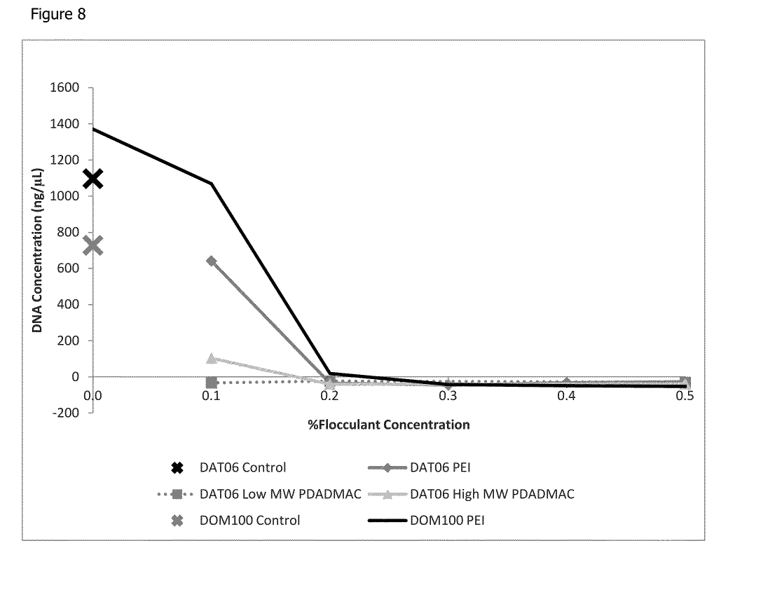

FIG. 8 shows the DNA concentration for the control and homogenised samples treated with the three types of flocculant. The presence of a substantial amount of DNA in the control, non-homogenised samples (crosses) suggests significant cell lysis has occurred. DOM100 control (grey cross) can be compared with DOM100 homogenised harvest with 0% PEI (black line) which indicates that approximately 50% of the cells have undergone autolysis. This is likely to increase the burden on the clarification steps. As can be seen the presence of the flocculants substantially reduces DNA concentration in the clarified harvest. In particular, the reduction in DNA concentration for the DOM100 harvest in the presence of PEI corresponds to the decreased turbidity (Example 3) and the improved primary filter train (Example 5), that has been correlated with the 5% or less particles in the ≦5 μm range as shown in Table 2, and in particular, the optimal sweet spot at the PEI concentration of 0.3-0.5%.

This Example also shows that the results for two alternative flocculants (high or low MW PDADMAC) are comparable with that of PEI.

### Example 7

DOM101 harvest was centrifuged as in Example 4 to create centrate in the presence and absence of 0.5% PEI. The volume of filtrate that was achieved on a small scale filter containing a Pall Seitz-EKS 60D 0.2 μm filter (depth filter with nominal pore size 0.05-0.2 μm) prior to blocking was then measured and plotted against time for both samples using a vacuum driven small scale system on the Tecan Evo II (Tecan, Theale, UK).

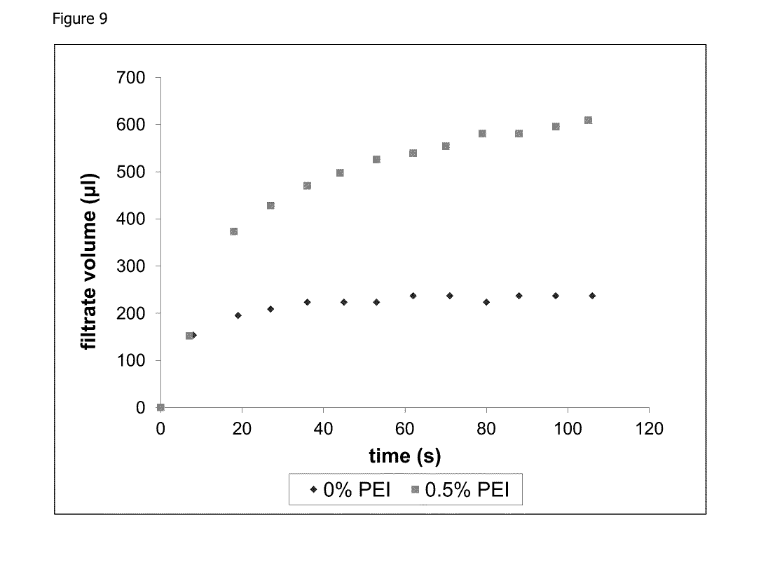

FIG. 9 shows that in the presence of 0.5% PEI the filtrate volume achievable is almost 3 times that achievable without PEI—with 0% PEI the maximum is achieved at 200 μl filtrate volume in 30 s and with 0.5% PEI this is still rising slowly at 600 μl in 110 s. This has a significant effect on the filterability of the DOM101 harvest and a subsequent reductive effect on the cost of such a process.

### Example 8

DOM101 was harvested at various times post induction, and half the samples treated with 0.5% PEI. Both the PEI and non-PEI treated samples were then centrifuged as in Example 4 and then subjected to filtration studies as in Example 7. Vₘₐₓ was then calculated for both sets of samples and plotted against induction time. The Vₘₐₓ measurement is a direct measurement of the filterability of the sample and can be used to scale up a filtration process based upon the data received.

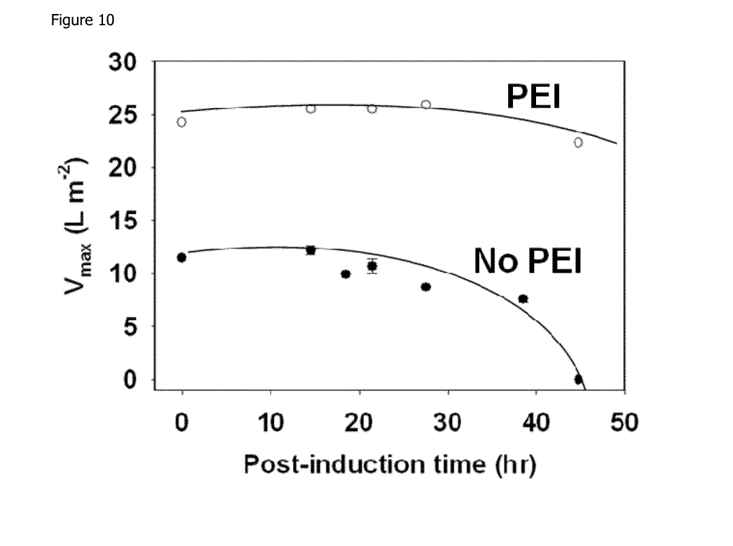

As can be seen in FIG. 10 the presence of a 0.5% PEI flocculation step in the process significantly improves the filterability by increasing the maximum achievable filtrate by 250% (0 hours post induction), increasing to 2500% by the end of the fermentation (45 hours post induction). It can be observed that at approximately 25 hour post induction the filterability of the centrate decreases dramatically in the non-PEI treated sampled to almost zero by the end of fermentation. The Vₘₐₓ for samples treated with PEI not only remain constantly higher but also are less susceptible to post-induction time showing that a 0.5% PEI flocculation step adds considerable robustness to a clarification process.

The decrease in filterability at the post induction time of 25 hours can be associated with the amount of auto-lysis observed in the fermentation cell broth which can be approximately 50% (see Example 6 and Example 9 below).

### Example 9

Auto-lysis can also be indirectly measured using a capacitance probe (Aber Instruments Ltd, Aberystwyth, UK), which measures the percentage decrease in capacitance from the maximum measurement recorded during the fermentation to the troph (lowest point) after the maximum measurement is calculated, which is usually the same as at harvest. Table 4 demonstrates the amount of cell lysis observed in a number of DOM101 fermentation replicates as measured by capacitance.

TABLE 4
variation in cell lysis as determined by capacitance in DOM101 harvests

| | Proportion of cell |
|---|---|
| DOM101 fermentation | lysis observed at harvest (%) |
| A | 30 |
| B | 32 |
| C | 41 |
| D | 24 |
| E | 19 |
| F | 20 |

### Example 10

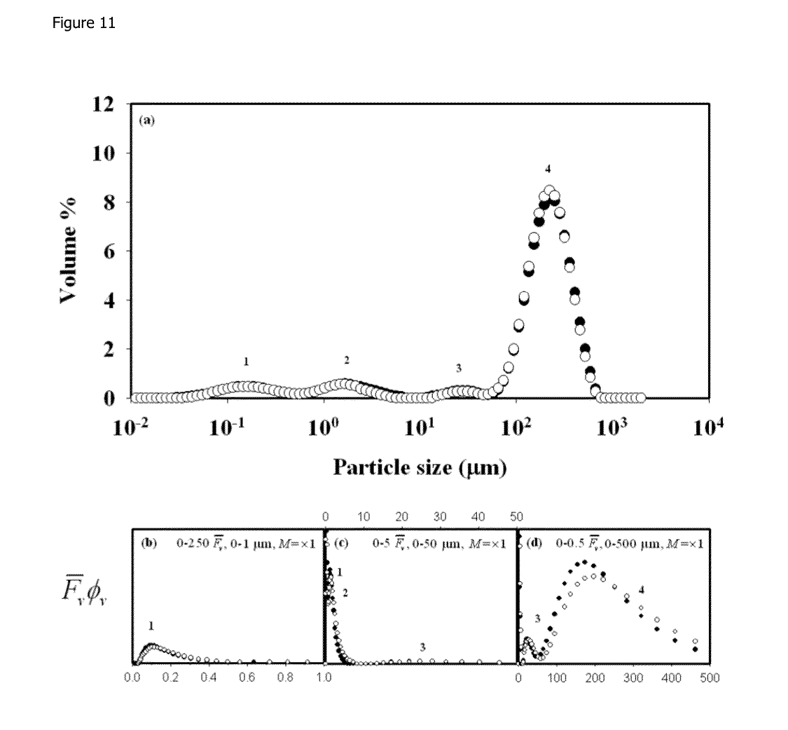

FIG. 11 demonstrates the properties and effect of shear on frozen and thawed (thawed) harvest expressing DOM101. Particle size distributions (calculated as in Example 1) are presented for thawed harvest (open circle), and for thawed harvest subjected to high shear at εmax=0.53×10⁶ W kg⁻¹ as in Example 2 (closed circle). The relative solids volume fraction, φv, is 0.11 w/v for thawed harvest and for thawed harvest subjected to high shear. Volume ratio of peaks 1, 2, 3, 4 are 5:7:4:84 for both materials.

As can be seen the distributions are very similar for the thawed material. Table 5, shows the % volume of sample particles ≦5 μm diameter by total volume, which shows 13.2% for non-sheared and 12.3% for sheared. When compared with FIG. 3 which shows the effect of shear on harvest that has not been pre-treated, it appears that the freeze-thaw process has a stabilising effect on the particle size distribution of the samples studied in the presence of high shear. This is an interesting observation for experimental material, however in bio-processing it is less likely that material would be frozen as part of clarification.

### Addition of Flocculant

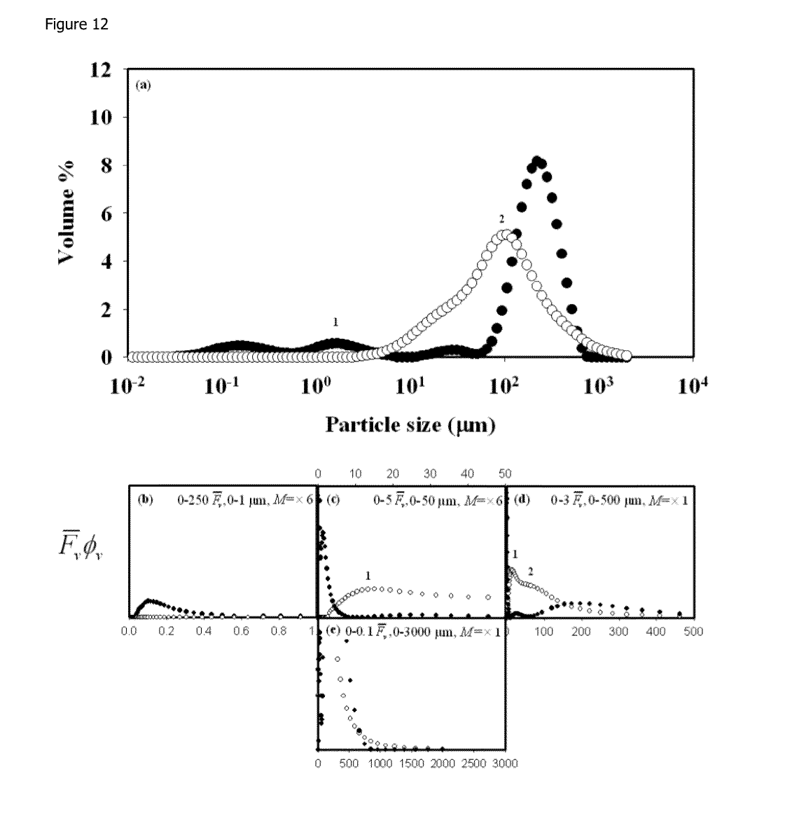

FIG. 12 shows the effect of 0.5% PEI flocculation on freeze-thawed harvest expressing DOM101. Particle size distributions are presented for thawed harvest (closed circle), and PEI-flocculated thawed harvest subjected to high shear at εmax=0.53×106 W kg⁻¹ as in Example 2 (open circle). The relative solids volume fraction, φv, are 0.11 w/v for thawed harvest and 0.15 w/v for PEI flocculated material (φv values quoted are corrected for dilution factor with PEI solution). The volumes ratios of peak 1 and 2 are ˜20:80.

As can be seen from Table 5, the percentage of ≦5 μm particles decreases after the PEI is added from 8.08% to 0.6%.

### Example 11

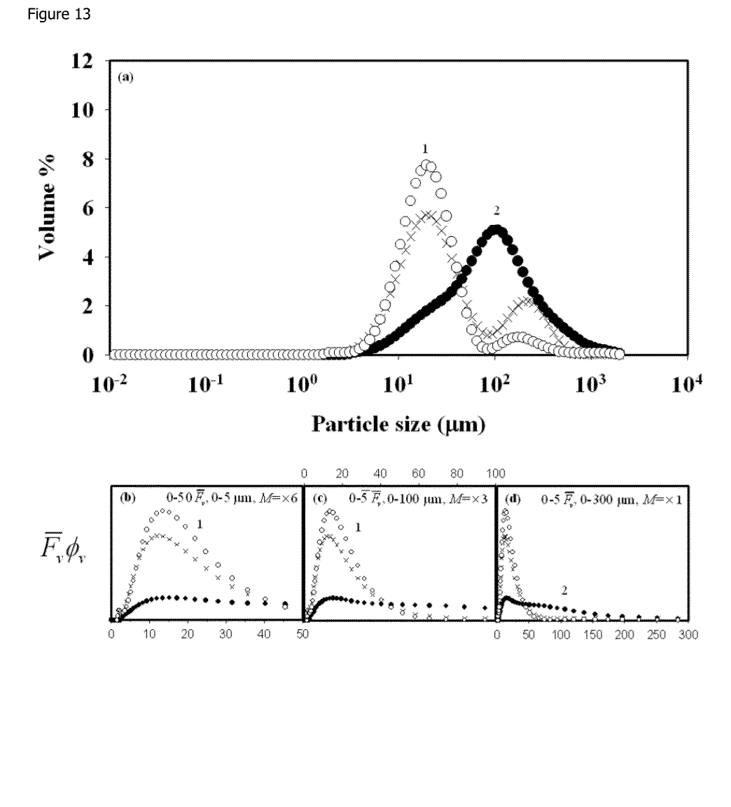

FIG. 13 show the effect of low and high shear on PEI flocculated freeze-thawed harvest, expressing DOM101. Particle size distributions (measured as in Example 1) are presented for PEI flocculated thawed harvest (closed circle) and for PEI flocculated thawed harvest subjected to low shear at εmax of 0.04×10⁶ W kg⁻¹ (cross) and high shear at εmax of 0.53×10⁶ W kg⁻¹ (open circle) as in Example 2. The relative solids volume fraction, φv, are 0.13 w/v for PEI flocculated thawed harvest with low shear and 0.12 w/v for PEI flocculated thawed harvest with high shear (φv values quoted are corrected for dilution factor with PEI solution).

As can be seen from Table 5, the effect of shear on the PEI flocculated thawed harvest is to reduce the size of the particles present, yet the presence of PEI maintains the majority of the particles above the ≦5 μm range (the % distribution shifts from 0.6% to 2.01% (low shear) or to 1.84% (high shear). This shows that the PEI clarification step is a robust step even in the presence of increasing levels of shear.

### Example 12

Freeze-thawed harvest expressing DOM101 was subjected to either shear or homogenisation using a high pressure homogeniser (Gaulin Micron Lab40, Lubeck, Germany) operated at 500 bar and 4° C. for 2 passes. Particle size distributions were then determined for the samples as measured in Example 1.

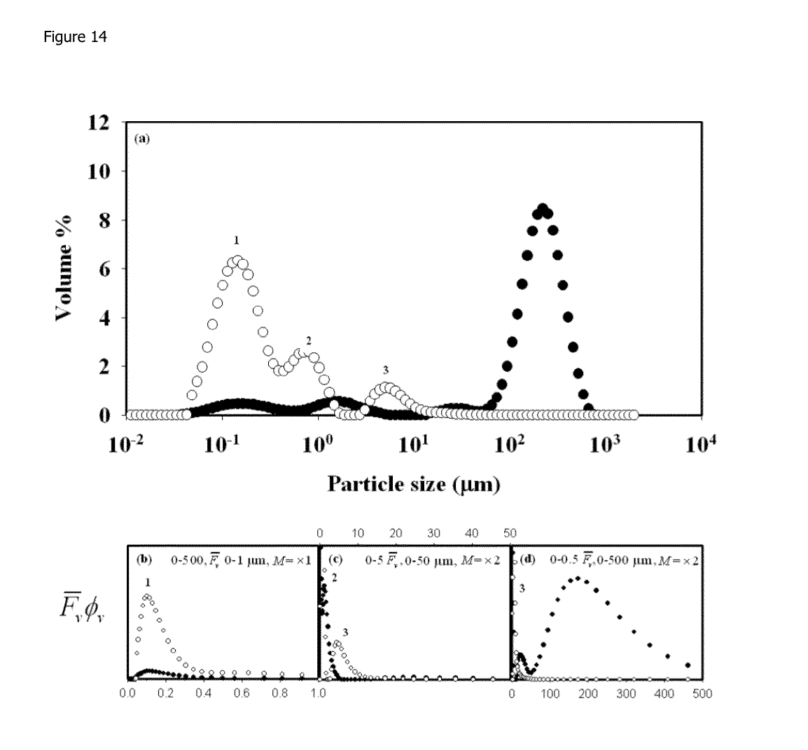

FIG. 14 shows the effect of homogenisation on the particle size distribution. Particle size distributions are presented for sheared thawed harvest (closed circle) and for homogenised harvest (open circle). The relative solids volume fractions, φv, are 0.11 w/v for thawed harvest and 0.078 w/v for homogenised harvest.

As can be seen from the distributions in FIG. 14 and Table 5, homogenisation has a dramatic impact on the particle size distribution of the thawed harvest, with the number of particles in the ≦5 μm range rising to 94.73%. The prevalence of the very small particles in the homogenised sample would have an extremely detrimental effect on bio-processing.

TABLE 5
Percentage volume of particles ≦5 μm in diameter under various
conditions described in Examples 10, 11 and 12.

| | % volume |
|---|---|
| | of particles ≦5 μm |
| Sample | diameter by total volume |
| (FIG. 11) DOM101 thawed harvest | 13.22 |
| (FIG. 11) DOM101 sheared thawed harvest | 12.32 |
| (FIG. 12) DOM101 thawed harvest | 8.08 |
| (FIG. 12) DOM101 sheared thawed harvest | 0.6 |
| with 0.5% PEI treatment | |
| (FIG. 13) DOM101 thawed harvest with 0.5% | 0.6 |
| PEI treatment | |
| (FIG. 13) DOM101 thawed harvest with 0.5% | 2.01 |
| PEI treatment, low shear | |
| (FIG. 13) DOM101 thawed harvest with 0.5% | 1.84 |
| PEI treatment, high shear | |
| (FIG. 14) DOM101 sheared thawed harvest | 13.22 |
| (FIG. 14) DOM101 thawed homogenised | 94.73 |
| harvest | |

### Example 13

Dat01 harvest was homogenised using a Gaulin-type homogenized at a target pressure of 10,000 psi for 2 passes. The homogenised harvests were treated with increasing concentrations of PEI. Table 6 below shows that the large percentage of particles in the ≦5 μm range can be reduced to less than 5% by the addition of 0.054%-0.99% or 0.374%-0.65% PEI (upper limit tested). Thus, the pre-treatment conditioning step of homogenisation can also benefit from the appropriate amount of PEI addition to result in a more efficient clarification process.

TABLE 6
Percentage volume of particles under 5 μm in diameter with increasing
levels of PEI for a protein homogenate.

| PEI Concentration | % volume of Dat01 homogenate particles |
|---|---|
| % w/v | ≦5 μm diameter by total volume |
| 0 | 81.78 |
| 0.054 | 3.27 |
| 0.99 | 4.53 |
| 0.146 | 5.49 |
| 0.194 | 6.77 |
| 0.244 | 6.98 |
| 0.374 | 2.82 |
| 0.509 | 2.44 |
| 0.65 | 2.14 |

### Example 14

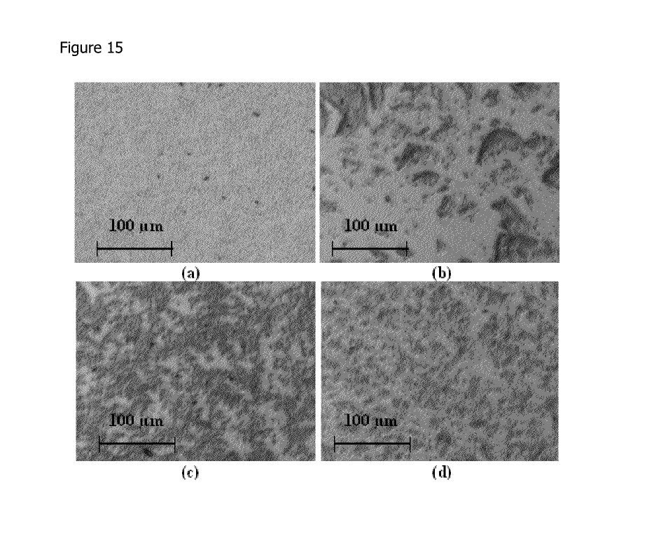

Freeze-thawed DOM101 harvest images were captured using conventional microscopy prior to (a) and after addition of 0.5% PEI (b) and subjected to either low (c) or high (d) shear as described above, shown in FIG. 15.

As can be seen from FIG. 15, a substantial amount of flocs of irregular large size are formed upon addition of PEI (b). These then become somewhat smaller and more even upon subjection to low shear (c) and more so upon high shear (d). The flocs present upon high shear are larger than the cells observed in the untreated image (a).

### Example 15

Freeze-thawed DOM101 harvests were subjected to ultra-scale down centrifugation studies in the same manner as was performed in Example 4. Thawed harvest samples were subjected to 0.5% PEI flocculation (c). Thawed harvest samples were also subjected to homogenisation using a high pressure homogeniser (Gaulin Micron Lab40, Lubeck, Germany) operated at 500 bar and 4° C. for 2 passes (a). The different suspensions were all exposed to conditions of: no shear (filled circle); low shear (open circle); high shear (open triangle) (as described above).

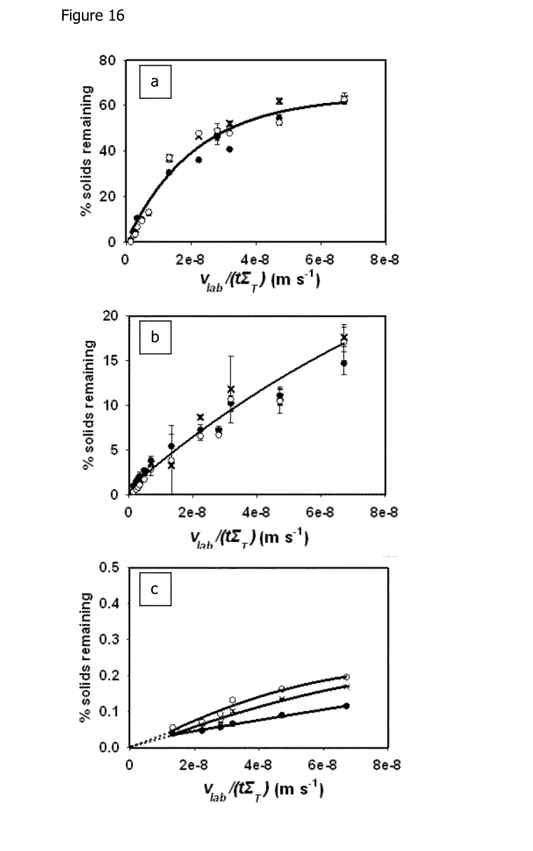

FIG. 16 shows percentage solids remaining for: (a) homogenised thawed harvest, (b) thawed harvest, and (c) PEI flocculated thawed harvest. Data presented as mean±s.d.; lines are best least square fit using 3rd order polynomials. For graphs (a) and (b) single correlations are given as there is no consistent trend with increasing shear rate. In all cases the correlations are fitted through origin which provides the control.

As can be seen from the Figures the thawed samples (b) show up to 16% solids remaining and the homogenised samples (a) show up to 60% solids remaining—neither of these are suitable for further processing as the % solids remaining are too high—typically the desired amount is less than 1%. This target of less than 1% is achieved comfortably with the addition of 0.5% PEI, which shows a reduction to less than 0.2% solids remaining.

### Example 16

No significant difference was observed in the yield of DOM101 from DOM101 harvest, or in the profile of monomer/dimer for any of the samples described above (data not shown here). It is assumed that this is also true of the other recombinant proteins described.

### Example 17

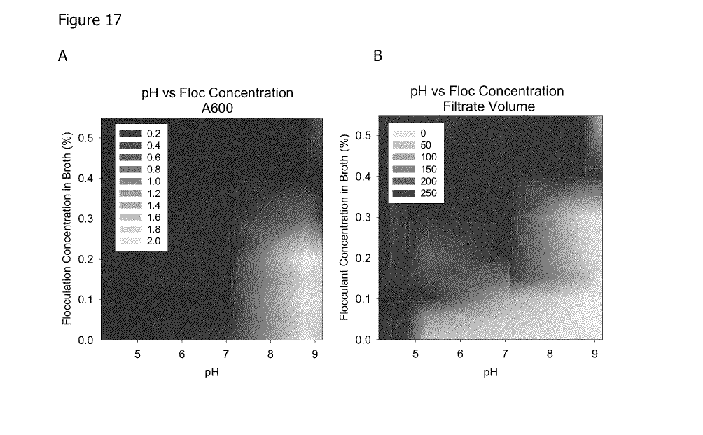

The pre-prepared 1.5% PEI solution was added to the fermentation harvest (Dat06) to give the desired concentration range of PEI (0 to 0.6%). The pH of the solution was adjusted with 200 mM Acetic acid or 1M NaOH to achieve the desired pH range (4 to 9). The pH of a typical cell broth is between pH6-7. After approximately 5-10 minutes of mixing at room temperature, flocculated particulates from each PEI concentration and pH condition were separated from the supernatant using a batch centrifuge at 3400 rcf for 20 minutes to complete flocculant settling. The resulting supernatant turbidity was measured at 600 nm wavelength to assess solution clarity with results shown in FIG. 17(A). The processibility was measured by direct filtration performance through a 0.2 μm filter under a centrifuge force of 3400 rcf for 90 seconds, with results shown in FIG. 17(B). While particle size was not measured directly for these flocculation conditions, correlations between clarity and filtration performance with particle size distribution, have been established in FIGS. 2, 5 and 7. The use of a plate format with 0.2 μm filter and absorbance readings can be used as a high throughput format to gain understanding of a design space.

The pH in addition to the flocculant concentration may influence the flocculation behaviour of E coil solutions. This Example shows that the interaction of pH and flocculant concentration had an effect on the clarity of the solution. At a flocculant concentration of >0.4% PEI, the turbidity of the solution is low regardless of the solution pH. Below a flocculant concentration of 0.3% PEI the solution clarity is greater (i.e. low turbidity) below a pH of 7.0. The results of this study are in line with the more detailed particle size analysis shown in FIG. 1; suggesting that pH in combination with PEI concentration may be used to fine tune the number of particles below 5 μm in size.

### Example 18

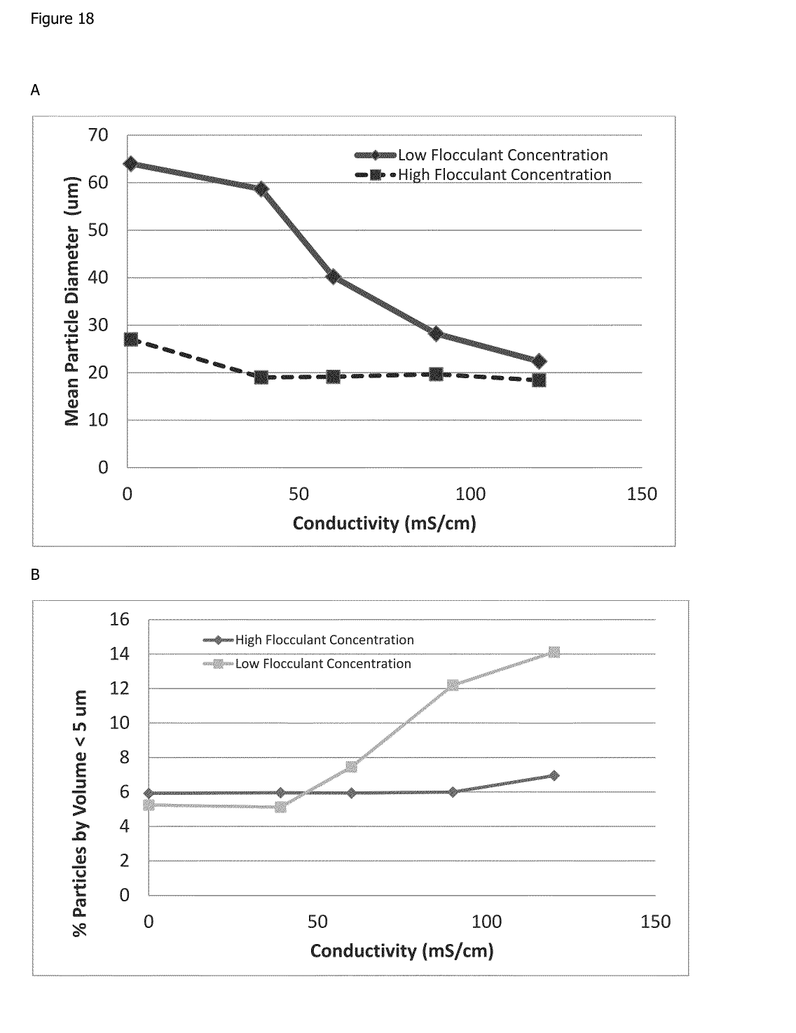

Harvest samples of Dat06 were treated with 0.1% PEI and 0.4% PEI as outlined in Example 1. The term “low flocculant” is used to represent 0.1% PEI, and the term “high flocculant” to represent 0.4% PEI in FIG. 18, for comparative reasons. The particle sizes of the two concentrations of PEI were determined as in previous examples for the “0” conductivity samples. For the conductivity samples, the diluent was changed from pure water to NaCl solutions of varying ionic strength. The results are shown in FIG. 18 for the mean particle diameter (A), and the % particles ≦5 μm by volume (B). All samples were taken from the same fermentation broth, but placed in different salt solutions at a dilution level of >1:100.

The 0.1% PEI treated sample placed in a water matrix had a much larger mean particle diameter than the sample treated with a higher concentration of PEI at 0.4%. At high salt (NaCl) concentrations the mean particle diameter for the different flocculant concentration became more similar. For the “low flocculant concentration” 0.1% PEI, the mean particle diameter is higher than at the “high flocculant concentration” 0.4% PEI, for low levels of conductivity (and subsequently) ionic strength. At higher concentrations of salt (high conductivity and ionic strength) mean particle diameter is much less variable for different levels of flocculant concentration. This allows for the fine tuning of mean particle diameter based on salt concentration as well as flocculant concentration. FIG. 18A shows an example of this phenomenon with mean particle diameter reaching greater than 60 μm for 0.1% flocculant and low conductivity and an average for 20-30 μm particle size when the conductivity is greater than 100 mS/cm with the mean particle diameter being much less sensitive to flocculant concentration at high ionic strength.

For a “low flocculant concentration” 0.1% PEI the volume of particles ≦5 μm in size increases at high conductivities, while the particles ≦5 μm in size stays relatively similar for the “high flocculant concentration” 0.4% PEI over a wide range of conductivities. Observations on both the mean particle diameter and the particles ≦5 μm in size support a more stable floc at the “high flocculant concentration” 0.4% PEI for Dat06. Both concentrations of PEI (0.1% and 0.4%) achieve the “about 5%” population of ≦5 μm at 0 conductivity, but the higher concentration of PEI (0.4%) achieves a more stable % of ≦5 μm population over the increasing conductivity.

### Example 19

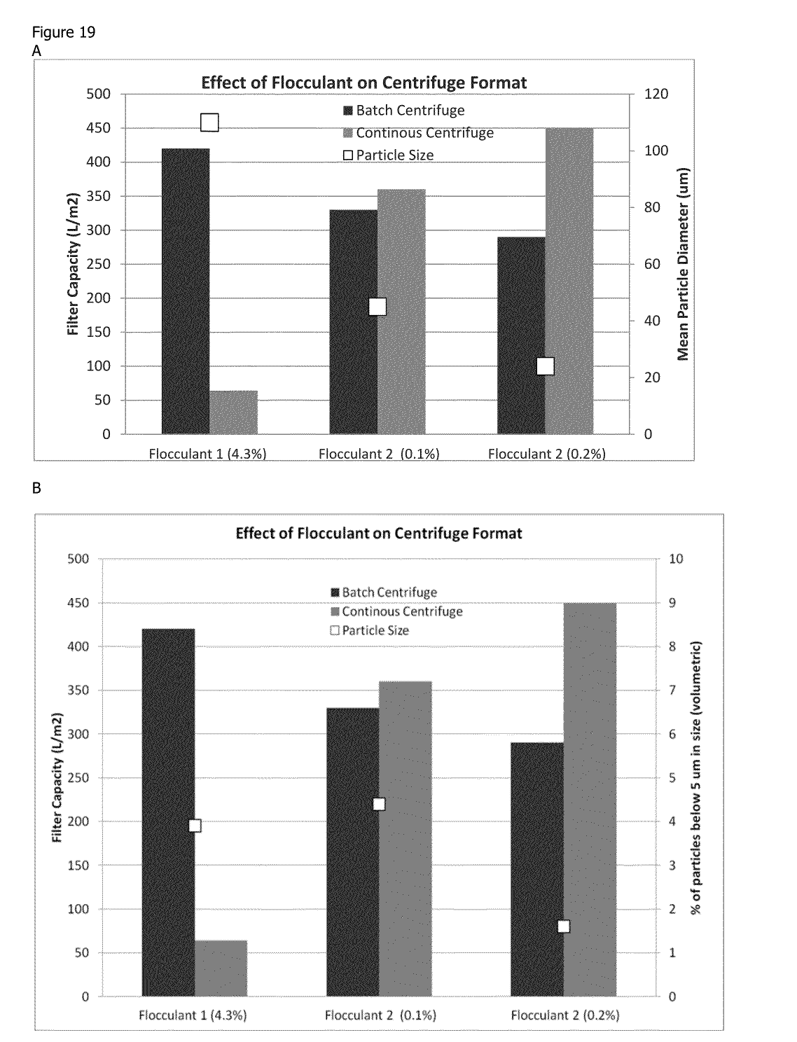

DOM100 harvest was flocculated with 4.3% CaCl₂, 0.1% PEI and 0.2% PEI by adding each component and mixing for approximately 1 hour; similar to the procedure followed in Example 1. The average particle size of the fermentation broth was then measured by static light scattering (Malvern Mastersizer). Samples were split into two separate aliquots; one was batch centrifuged and the other was centrifuged using a tubular bowl centrifuge (continuous centrifuge); both with similar total acceleration force. The resulting supernatant from the centrifuge samples were then filtered using a depth/membrane filter train at a constant flow rate to remove remaining cell debris. Filter capacity was measured by dividing the total volume processed prior to reaching 25 psi back pressure by the frontal area of the primary depth filter. The majority of particles in all cases were >5 μm in size. Mean particle diameter was assessed in FIG. 19A, and % particles ≦5 μm by volume in FIG. 19B.

Samples with a smaller average particle size as measured by static light scattering had a lower primary depth filter capacity of the batch centrifuged samples. This correlation suggests that larger average size particles formed during flocculation followed by a batch centrifugation will improve the filter capacity of the subsequent depth filters. The opposite result is observed for the samples processed through the Carr tubular bowl centrifuge. If the number of particles ≦5 μm size limit is compared to the batch centrifuge performance, it is observed that the performance was correlated.

### Example 20

Dat06 and DOM100 harvests were treated as described in Example 1. Samples with 0.4% PEI addition were compared to the samples that were not treated with a flocculant. Clarification was then performed by centrifugation and HCP levels were measured using in house analytical immunoassays.

Large levels of HCP species can be considered to be an indicator of cell lysis. Large increases can indicate significant quantities of cell lysis, which may cause viscosity increases and difficulties with clarification. High HCP levels may also cause additional downstream purification challenges.

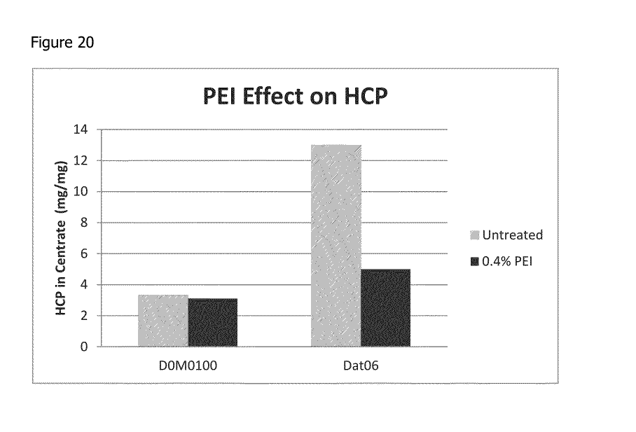

FIG. 20 shows the HCP concentration for the DOM100 and Dat06 samples with and without treatment of 0.4% PEI. While PEI is able to remove a substantial amount of the host cell protein population in Dat06 this is not the case for DOM100. The result exemplifies the complex nature of a host cell protein population and the difference that may be expected across products. The PEI may be able to remove a base level of HCPs and/or the flocculant may be able to remove specific types of host cell proteins more effectively than others.

Sequence Listing
SEQ ID NO: 1
Amino acid sequence of dAt01 (Exendin4-Dom7h-14-10 AlbudAb)
HGEGTFTSDLSKQMEEEAVRLFIEWLKNGGPSSGAPPPSGGGGGSGGGGSGGGGSDIQMTQSPSSLSASVGDR
VTITCRASQWIGSQLSVVYQQKPGKAPKLLIMWRSSLQSGVPSRFSGSGSGTDFTLTISSLQPEDFATYYCAQGL
RHPKTFGQGTKVEIKR
SEQ ID NO: 2
DNA sequence of dAt01 (Exendin4-Dom7h-14-10 AlbudAb) - (no signal sequence)
CACGGTGAAGGTACGTTCACCTCTGACCTGAGCAAACAGATGGAGGAAGAAGCGGTTCGTCTGTTCATCGAG
TGGCTGAAAAACGGTGGTCCGTCTTCTGGTGCTCCGCCGCCGTCTGGTGGTGGTGGTGGTTCTGGTGGTGG
TGGTTCTGGTGGTGGTGGCAGCGATATCCAGATGACTCAGTCCCCGTCTTCTCTCTCCGCCTCTGTTGGCGA
CCGTGTTACCATCACTTGTCGTGCGAGCCAGTGGATCGGTTCCCAGCTGAGCTGGTATCAGCAGAAACCGGG
CAAAGCGCCGAAACTGCTGATCATGTGGCGCTCTAGCCTGCAGTCTGGTGTACCGTCTCGTTTCTCCGGCTC
TGGTTCTGGTACGGACTTCACCCTCACGATCTCTTCCCTGCAGCCGGAAGACTTTGCCACCTACTACTGCGCA
CAGGGTCTGCGTCACCCGAAAACCTTCGGTCAGGGTACCAAAGTCGAGATCAAACGT
SEQ ID NO: 3
Amino acid sequence of dAt06 (Dom7h-11-15 R108C) AlbudAb
DIQMTQSPSSLSASVGDRVTITCRASRPIGTMLSVVYQQKPGKAPKLLILAFSRLQSGVPSRFSGSGSGTDFT
LTISSLQPEDFATYYCAQAGTHPTTFGQGTKVEIKC
SEQ ID NO: 4
DNA sequence of dAt06 (Dom7h-11-15 R108C) AlbudAb - (no signal sequence)
GATATCCAGATGACCCAGTCTCCGTCTTCCCTGTCTGCGTCTGTTGGTGATCGCGTTACCATCACTTGCCGT
GCAAGCCGTCCGATCGGTACTATGCTGAGCTGGTACCAGCAGAAACCGGGTAAAGCGCCGAAACTGCTGATT
CTGGCTTTCTCTCGCCTGCAGTCTGGTGTTCCGTCTCGTTTCAGCGGTAGCGGTTCTGGTACCGACTTCACC
CTGACCATTTCCTCTCTGCAGCCGGAAGACTTCGCTACCTACTATTGTGCGCAGGCAGGTACTCACCCGACTA
CCTTCGGTCAGGGCACCAAAGTTGAAATCAAATGC
SEQ ID NO: 5
Amino acid sequence of DOM100 (DMS5541) AlbudAb - TNFR1
EVQLLESGGGLVQPGGSLRLSCAASGFTFDKYSMGWVRQAPGKGLEVVVSQISDTADRTYYAHAVKGRFTISRD
NSKNTLYLQMNSLRAEDTAVYYCAIYTGRWVPFEYWGQGTLVIVSSASTDIQMTQSPSSLSASVGDRVTITCRA
SRPIGTTLSWYQQKPGKAPKLLILWNSRLQSGVPSRFSGSGSGTDFTLTISSLQPEDFATYYCAQAGTHPTTFGQ
GTKVEIKR
SEQ ID NO: 6
DNA sequence of DOM100 (DMS5541) AlbudAb - TNFR1 - (no signal sequence)
GAGGTACAGCTGCTGGAATCTGGTGGTGGTCTGGTTCAGCCGGGTGGCTCTCTGCGTCTGTCTTGTGCAGC
GTCTGGTTTCACCTTCGACAAATACTCTATGGGCTGGGTTCGTCAGGCGCCGGGTAAAGGTCTGGAATGGGT
GTCTCAGATCTCTGACACCGCAGATCGTACCTACTACGCACACGCTGTGAAAGGTCGCTTCACCATCTCTCGC
GACAACTCCAAAAACACCCTGTACCTGCAGATGAACTCCCTGCGTGCTGAAGACACCGCGGTATACTATTGC
GCGATCTACACCGGTCGTTGGGTTCCGTTCGAATACTGGGGTCAGGGTACCCTGGTTACTGTGAGCTCTGCG
TCTACCGACATCCAGATGACCCAGTCTCCGTCTTCTCTGTCTGCGAGCGTTGGTGACCGTGTTACCATCACTT
GCCGTGCTTCTCGTCCGATCGGTACCACTCTGAGCTGGTATCAGCAGAAACCGGGCAAAGCGCCGAAACTGC
TGATCCTGTGGAACTCTCGTCTGCAGTCCGGTGTTCCGTCTCGTTTCTCTGGCAGCGGTTCTGGTACCGACT
TCACCCTGACTATCTCTAGCCTGCAGCCGGAAGACTTCGCAACCTACTATTGCGCACAGGCTGGTACTCACCC
GACCACTTTCGGTCAGGGTACCAAAGTAGAAATCAAACGT
SEQ ID NO: 7
Amino acid sequence of DOM101
EVQLLESGGGLVQPGGSLRLSCAASGFTFAHETMVWVRQAPGKGLEWVSHIPPDGQDPFYADSVKGRFTISRD
NSKNTLYLQMNSLRAEDTAVYHCALLPKRGPWFDYWGQGTLVTVSS
SEQ ID NO: 8
DNA sequence of DOM101 - (no signal sequence)
GAAGTACAACTGCTGGAGAGCGGTGGCGGCCTGGTTCAACCGGGTGGTTCCCTGCGCCTGTCCTGTGCGGC
ATCTGGTTTCACCTTCGCACACGAAACCATGGTGTGGGTTCGCCAAGCTCCGGGCAAAGGCCTGGAATGGGT
AAGCCACATTCCTCCAGATGGCCAGGACCCATTCTATGCGGATTCCGTTAAGGGTCGCTTTACCATTTCTCGT
GATAACTCCAAAAACACCCTGTACCTGCAGATGAACTCCCTGCGCGCCGAGGATACTGCGGTGTACCATTGT
GCGCTGCTGCCTAAACGTGGCCCGTGGTTCGATTACTGGGGTCAGGGTACTCTGGTCACCGTAAGCAGC
SEQ ID NO: 9
Amino acid sequence of alanine extended DOM101
EVQLLESGGGLVQPGGSLRLSCAASGFTFAHETMVWVRQAPGKGLEVVVSHIPPDGQDPFYADSVKGRFTISRD
NSKNTLYLQMNSLRAEDTAVYHCALLPKRGPWFDYWGQGTLVTVSSA
SEQ ID NO: 10
DNA sequence of alanine extended DOM101 - (no signal sequence)
GAAGTACAACTGCTGGAGAGCGGTGGCGGCCTGGTTCAACCGGGTGGTTCCCTGCGCCTGTCCTGTGCGGC
ATCTGGTTTCACCTTCGCACACGAAACCATGGTGTGGGTTCGCCAAGCTCCGGGCAAAGGCCTGGAATGGGT
AAGCCACATTCCTCCAGATGGCCAGGACCCATTCTATGCGGATTCCGTTAAGGGTCGCTTTACCATTTCTCGT
GATAACTCCAAAAACACCCTGTACCTGCAGATGAACTCCCTGCGCGCCGAGGATACTGCGGTGTACCATTGT
GCGCTGCTGCCTAAACGTGGCCCGTGGTTCGATTACTGGGGTCAGGGTACTCTGGTCACCGTAAGCAGCGC
G

1. A method of producing a recombinant protein, wherein the method comprises:
(a) harvesting a microbial cell broth that expresses the recombinant protein; and
(b) adding an amount of a flocculant to achieve a particle size distribution by volume of about 5% or less particles in the size range of 5 μm or less.

2. The method of claim 1, wherein the method further comprises step:
(c) clarifying the flocculated harvest.

3. The method of claim 2, wherein the method further comprises step:
(d) purifying the recombinant protein from the clarified flocculated harvest.

4. A method of clarifying a microbial harvest, wherein the method comprises:
(a) harvesting a microbial cell broth;
(b) adding an amount of a flocculant to achieve a particle size distribution by volume of about 5% or less particles in the size range of 5 μm or less; and
(c) clarifying the flocculated harvest.

5. The method of claim 4, wherein the microbial cell broth expresses a recombinant protein.

6. The method of claim 2, wherein step (c) comprises at least one selected from the group consisting of (i) settling; (ii) centrifugation; and (iii) filtration.

7. The method of claim 1, wherein the expressed recombinant protein comprises a signal sequence.

8. The method of claim 7, wherein the signal sequence is a periplasmic targeting signal sequence.

9. The method of claim 1, wherein the amount of the flocculant is added in an amount of between 0.01-5% by the volume of the harvest.

10. The method of claim 1, wherein the amount of the flocculant is added in an amount of between 0.01-2% by the volume of the harvest.

11. The method of claim 1, wherein the flocculant is polyethylenimine, or poly(diallyldimethylammonium chloride).

12. The method of claim 1, wherein the flocculant is CaCl₂.

13. The method of claim 1, wherein the microbial cell broth is an Escherichia coli cell broth.

14. The method of claim 1, wherein the recombinant protein is an antigen binding protein.

15. The method of claim 14 wherein the antigen binding protein comprises at least one selected from the group consisting of:
(a) a peptide-dAb fusion; (b) a dAb conjugate; (c) a dAb-dAb fusion; and (d) a naked dAb.

16. The method of claim 14 wherein the antigen binding protein comprises at least one selected from the group consisting of:
(a) the amino acid sequence shown in SEQ ID NO:1; (b) the amino acid sequence shown in SEQ ID NO:3; (c) the amino acid sequence shown in SEQ ID NO:5; (d) the amino acid sequence shown in SEQ ID NO:7; and (e) the amino acid sequence shown in SEQ ID NO: 9.

17. A modified Escherichia coli cell harvest wherein:
(a) the cells express a periplasmic targeted recombinant protein;
(b) the harvest comprises 0.01-2% polyethylenimine by volume; and
(c) the particle size distribution by volume of the harvest is about 5% or less particles in the size range of 5 μm or less.

18. The modified Escherichia coli cell harvest of claim 17, wherein the recombinant protein comprises an antigen binding protein.

19. The modified Escherichia coli cell harvest of claim 18, wherein the antigen binding protein comprises at least one selected from the group consisting of:
(a) a peptide-dAb fusion; (b) a dAb conjugate; (c) a dAb-dAb fusion; and (d) a naked dAb.

20. The modified Escherichia coli cell harvest of claim 18, wherein the antigen binding protein comprises at least one selected from the group consisting of:
(a) the amino acid sequence shown in SEQ ID NO:1; (b) the amino acid sequence shown in SEQ ID NO:3; (c) the amino acid sequence shown in SEQ ID NO:5; (d) the amino acid sequence shown in SEQ ID NO:7; and (e) the amino acid sequence shown in SEQ ID NO: 9.
